(19) United States
(12) Patent Application Publication
(10) Pub. No.: US 2024/0317764 A1
(43) Pub. Date: Sep. 26, 2024

(54) SALTS AND SOLID FORMS OF A COMPOUND HAVING APJ RECEPTOR ACTIVITY

(71) Applicant: Annapurna Bio, Inc., South San Francisco, CA (US)

(72) Inventors: Libo CHEN, Shanghai (CN); Xinglong JIANG, Hillsborough, NJ (US)

(21) Appl. No.: 18/620,951

(22) Filed: Mar. 28, 2024

Related U.S. Application Data
(63) Continuation of application No. PCT/CN2023/130583, filed on Nov. 9, 2023.

(30) Foreign Application Priority Data
Nov. 10, 2022 (WO) PCT/CN2022/131179

(51) Int. Cl. C07D 487/04; A61K 31/4985; C07C 215/40

(52) U.S. Cl. CPC C07D 487/04; A61K 31/4985; C07C 215/40; C07B 2200/13

(57) ABSTRACT

The present disclosure relates to salts and solid forms of a compound that modulates APJ receptor activity, and their use as a therapeutic agent for treating diseases disorders, or conditions associated with repressed or impaired APJ receptor signaling, such as pulmonary arterial hypertension (PAH).

### CROSS REFERENCE TO RELATED APPLICATIONS

This application claims the benefit of International Patent Application Number PCT/CN2022/131179 filed on Nov. 10, 2022, the contents of which are hereby incorporated herein in its entirety.

### FIELD

The present disclosure relates generally to salts and solid forms of a compound that modulate APJ receptor activity, pharmaceutical compositions thereof, therapeutic uses thereof, and processes for making the same.

### BACKGROUND

The present disclosure relates to salts and solid forms of a compound that modulates apelin (APJ) receptor activity, and its use as a therapeutic agent for treating diseases disorders, or conditions associated with repressed or impaired APJ receptor signaling, such as pulmonary arterial hypertension (PAH).

### SUMMARY

The present disclosure provides salts and solid forms of Compound I (CAS Registry Number: 2415197-87-2), and co-crystals and solvates thereof. Also described herein are processes for making salts and solid forms of Compound I, pharmaceutical compositions comprising salts or solid forms of Compound I, and methods for using the same, in the treatment of diseases mediated by APJ receptor activity.

### BRIEF DESCRIPTION OF THE DRAWINGS

FIG. 1A shows an X-ray powder diffraction (XRPD) of Compound I choline salt Form A.

FIG. 1B shows a thermogravimetric analysis (TGA)/differential scanning calorimeter (DSC) curve of Compound I choline salt Form A.

FIG. 2A shows an X-ray powder diffraction (XRPD) of Compound I choline salt Form B.

FIG. 2B shows a thermogravimetric analysis (TGA)/differential scanning calorimeter (DSC) curve of Compound I choline salt Form B.

FIG. 3A shows an X-ray powder diffraction (XRPD) of Compound I free acid Form A.

FIG. 3B shows thermogravimetric analysis (TGA)/differential scanning calorimeter (DSC) curve of Compound I free acid Form A.

FIG. 4A shows an X-ray powder diffraction (XRPD) of Compound I free acid Form B.

FIG. 4B shows a thermogravimetric analysis (TGA)/differential scanning calorimeter (DSC) curve of Compound I free acid Form B.

FIG. 5A shows an X-ray powder diffraction (XRPD) of Compound I free acid Form C.

FIG. 5B shows a thermogravimetric analysis (TGA)/differential scanning calorimeter (DSC) curve of Compound I free acid Form C.

FIG. 6A shows an X-ray powder diffraction (XRPD) of Compound I sodium salt Form A.

FIG. 6B shows a thermogravimetric analysis (TGA)/differential scanning calorimeter (DSC) curve of Compound I sodium salt Form A.

FIG. 7A shows an X-ray powder diffraction (XRPD) of Compound I potassium salt Form A.

FIG. 7B shows a thermogravimetric analysis (TGA)/differential scanning calorimeter (DSC) curve of Compound I potassium salt Form A.

FIG. 8A shows an X-ray powder diffraction (XRPD) of Compound I potassium salt Form A and Compound I potassium salt Form B.

FIG. 8B shows a thermogravimetric analysis (TGA)/differential scanning calorimeter (DSC) curve of Compound I potassium salt Form B.

FIG. 9A shows an X-ray powder diffraction (XRPD) of Compound I calcium salt Form A.

FIG. 9B shows a thermogravimetric analysis (TGA)/differential scanning calorimeter (DSC) curve of Compound I calcium salt Form A.

FIG. 10A shows an X-ray powder diffraction (XRPD) of Compound I magnesium salt Form A.

FIG. 10B shows a thermogravimetric analysis (TGA)/differential scanning calorimeter (DSC) curve of Compound I magnesium salt Form A.

FIG. 11A shows an X-ray powder diffraction (XRPD) overlay of Compound I sodium salt Form A (top) and Compound I sodium salt Form B (bottom).

FIG. 11B shows a thermogravimetric analysis (TGA)/differential scanning calorimeter (DSC) curve of Compound I sodium salt Form B.

FIG. 12 shows a ORTEP drawing of the crystal structure of Compound I choline salt Form A.

### DETAILED DESCRIPTION

The compound N-(1-(2,6-dimethoxyphenyl)-2-(6-ethoxypyridin-2-yl)-1H-imidazo[4,5-b]pyrazin-6-yl)methanesulfonamide, designated herein as Compound I, has the following formula:

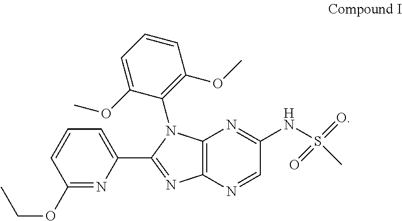 [SMILES: CCOc1cccc(-c2nc3ncc(NS(C)(=O)=O)nc3n2-c2c(OC)cccc2OC)n1]

Compound I is a modulator of APJ receptor activity. The synthesis and method of use thereof is described in PCT International Application Publication No. WO2020/073011 which is incorporated by reference herein in its entirety.

### 1. Definitions

As used in the present specification, the following words and phrases are generally intended to have the meanings as set forth below, except to the extent that the context in which they are used indicates otherwise.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±2.5%. In certain other embodiments, the term “about” includes the indicated amount ±1%. Also, to the term “about x” includes description of “x”.

Recitation of numeric ranges of values throughout the disclosure is intended to serve as a shorthand notation of referring individually to each separate value falling within the range inclusive of the values defining the range, and each separate value is incorporated in the specification as it were individually recited herein.

Forms of Compound I or salts, co-crystals, or solvates thereof are provided herein. In one embodiment, reference to a form of Compound I or a salt, co-crystal, or solvate thereof means that at least 50% to 99% (e.g., at least 50%, at least 55%, at least 60%, at least 65%, at least 70%, at least 75%, at least 80%, at least 85%, at least 90%, at least 95%, or at least 99%) of Compound I or a salt, co-crystal, or solvate thereof present in a composition is in the designated form. For instance, in one embodiment, reference to Compound I free acid Form A means that at least 50%, at least 55%, at least 60%, at least 65%, at least 70%, at least 75%, at least 80%, at least 85%, at least 90%, at least 95%, or at least 99% of Compound I, as the free acid, is present in a composition as Form A.

The term “solid form” refers to a type of solid-state material that includes amorphous as well as crystalline forms. The term “crystalline form” refers to polymorphs as well as solvates (e.g., hydrates), etc. The term “polymorph” refers to a particular crystal structure having particular physical properties such as X-ray diffraction, melting point, and the like.

The term “co-crystal” refers to a molecular complex of a compound disclosed herein and one or more non-ionized co-crystal formers connected via non-covalent interactions. In some embodiments, the co-crystals disclosed herein may include a non-ionized form of Compound I (e.g., Compound I free form) and one or more non-ionized co-crystal formers, where non-ionized Compound I and the co-crystal former(s) are connected through non-covalent interactions. In some embodiments, co-crystals disclosed herein may include an ionized form of Compound I (e.g., a salt of Compound I) and one or more non-ionized co-crystals formers, where ionized Compound I and the co-crystal former(s) are connected through non-covalent interactions. Co-crystals may additionally be present in anhydrous or solvated forms. In certain instances, co-crystals may have improved properties as compared to the parent form (i.e., the free molecule, zwitterion, etc.) or a salt of the parent compound. Improved properties can be increased solubility, increased dissolution, increased bioavailability, increased dose response, decreased hygroscopicity, increased stability, a crystalline form of a normally amorphous compound, a crystalline form of a difficult to salt or unsaltable compound, decreased form diversity, more desired morphology, and the like. Methods for making and characterizing co-crystals are known to those of skill in the art.

The term “co-crystal former” or “co-former” refers to one or more pharmaceutically acceptable bases or pharmaceutically acceptable acids disclosed herein in association with Compound I, or any other compound disclosed herein.

The term “solvate” refers to a complex formed by combination of solvent molecules with molecules or ions of the solute. The solvent can be an organic compound, an inorganic compound, or a mixture of both. As used herein, the term “solvate” includes a “hydrate” (i.e., a complex formed by combination of water molecules with molecules or ions of the solute), hemi-hydrate, channel hydrate, etc. Some examples of solvents include, but are not limited to, methanol, N,N-dimethylformamide, tetrahydrofuran, dimethylsulfoxide, and water. In general, the solvated forms are equivalent to unsolvated forms and are encompassed within the scope of the present disclosure.

The term “desolvated” refers to a Compound I form that is a solvate as described herein, and from which solvent molecules have been partially or completely removed. Desolvation techniques to produce desolvated forms include, without limitation, exposure of a Compound I form (solvate) to a vacuum, subjecting the solvate to elevated temperature, exposing the solvate to a stream of gas, such as air or nitrogen, or any combination thereof. Thus, a desolvated Compound I form can be anhydrous, i.e., completely without solvent molecules, or partially solvated wherein solvent molecules are present in stoichiometric or non-stoichiometric amounts.

The term “amorphous” refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (glass transition).

Any formula or structure given herein, including Compound I, is also intended to represent unlabeled forms as well as isotopically labeled forms of the compounds. It is understood that for any given atom, the isotopes may be present essentially in ratios according to their natural occurrence, or one or more particular atoms may be enhanced with respect to one or more isotopes using synthetic methods known to one skilled in the art. Thus, hydrogen includes for example ¹H, ²H, ³H; carbon includes for example ¹¹C, ¹²C, ¹³C, ¹⁴C; oxygen includes for example ¹⁶O, ¹⁷O, ¹⁸O; nitrogen includes for example ¹³N, ¹⁴N, ¹⁵N; sulfur includes for example ³²S, ³³S, ³⁴S, ³⁵S, ³⁶S, ³⁷S, ³⁸S; fluoro includes for example ¹⁷F, ¹⁸F, ¹⁹F; chloro includes for example ³⁵Cl, ³⁶Cl, ³⁷Cl, ³⁸Cl, ³⁹Cl; and the like.

As used herein, the terms “treat,” “treating,” “therapy,” “therapies,” and like terms refer to the administration of material, e.g., any one or more solid, crystalline or polymorphs of Compound I as described herein in an amount effective to prevent, alleviate, or ameliorate one or more symptoms of a disease or condition, i.e., indication, and/or to prolong the survival of the subject being treated.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

As used herein, the term “modulating” or “modulate” refers to an effect of altering a biological activity, especially a biological activity associated with a particular biomolecule such as APJ receptor activity. For example, an agonist or antagonist of a particular biomolecule modulates the activity of the APJ receptor by either increasing (e.g. agonist, activator), or decreasing (e.g. antagonist, inhibitor) the activity, of the biomolecule. Such activity is typically indicated in terms of an inhibitory concentration (IC₅₀) or excitation concentration (EC₅₀) of the compound for an inhibitor or activator, respectively.

As used herein, the term “composition” refers to a pharmaceutical preparation suitable for administration to an intended subject for therapeutic purposes that contains at least one pharmaceutically active compound, including any solid form thereof. The composition may include at least one pharmaceutically acceptable component to provide an improved formulation of the compound, such as a suitable carrier or excipient.

As used herein, the term “subject” or “patient” refers to a living organism that is treated with compounds as described herein, including, but not limited to, any mammal, such as a human, other primates, sports animals, animals of commercial interest such as cattle, farm animals such as horses, or pets such as dogs and cats.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

In the present context, the term “therapeutically effective” or “effective amount” indicates that the materials or amount of material is effective to prevent, alleviate, or ameliorate one or more symptoms of a disease or medical condition, and/or to prolong the survival of the subject being treated. The therapeutically effective amount will vary depending on the compound, the disorder or condition and its severity and the age, weight, etc., of the mammal to be treated. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art.

In some embodiments, the phrase “substantially shown in Figure” as applied to an X-ray powder diffractogram is meant to include a variation of ±0.2°2θ or ±0.1°2θ, as applied to DSC thermograms is meant to include a variation of ±3° Celsius, and as applied to thermogravimetric analysis (TGA) is meant to include a variation of ±2% in weight loss.

“Substantially pure form (of a polymorph),” in some embodiments, means that in the referenced material, at least 99.9% of the material is the referenced polymorph. “Substantially pure form (of a polymorph),” in some embodiments, means that in the referenced material, at least 99.5% of the material is the referenced polymorph. “Substantially pure form (of a polymorph),” in some embodiments, means that in the referenced material, at least 99% of the material is the referenced polymorph. “Substantially pure form (of a polymorph),” in some embodiments, means that in the referenced material, at least 98% of the material is the referenced polymorph. “Substantially pure form (of a polymorph),” in some embodiments, means that in the referenced material, at least 97% of the material is the referenced polymorph. “Substantially pure form (of a polymorph),” in some embodiments, means that in the referenced material, at least 96% of the material is the referenced polymorph. “Substantially pure form (of a polymorph),” in some embodiments, means that in the referenced material, at least 95% of the material is the referenced polymorph. In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction between the compound and other specified material can occur.

### 2. Salts and Forms of Compound I

As described generally above, the present disclosure provides salts and crystalline forms of the compound, N-(1-(2,6-dimethoxyphenyl)-2-(6-ethoxypyridin-2-yl)-1H-imidazo[4,5-b]pyrazin-6-yl)methanesulfonamide (hereinafter “compound” of “Compound I”), or a salt, co-crystal, or solvate thereof. Crystalline forms of Compound I or a salt, co-crystal, or solvate thereof, and other forms (e.g., amorphous forms) of Compound I or a salt, co-crystal, or solvate thereof are collectively referred to herein as “forms of Compound I.”

In some embodiments, Compound I is a free acid. In some embodiments, Compound I is a salt. In some embodiments, Compound I is a pharmaceutically acceptable salt. In some embodiments, Compound I is a solvate. In some embodiments, Compound I is a hydrate. In some embodiments, Compound I is an anhydrate.

In one embodiment, provided is a solid form of N-(1-(2,6-dimethoxyphenyl)-2-(6-ethoxypyridin-2-yl)-1H-imidazo[4,5-b]pyrazin-6-yl)methanesulfonamide (Compound I), or solvate thereof. In one embodiment, provided is a solid form of a salt of N-(1-(2,6-dimethoxyphenyl)-2-(6-ethoxypyridin-2-yl)-1H-imidazo[4,5-b]pyrazin-6-yl)methanesulfonamide (Compound I), or solvate thereof.

### Salts of Compound I

In one embodiment, provided is a salt of N-(1-(2,6-dimethoxyphenyl)-2-(6-ethoxypyridin-2-yl)-1H-imidazo[4,5-b]pyrazin-6-yl)methanesulfonamide (Compound I), or solvate thereof, having the formula:

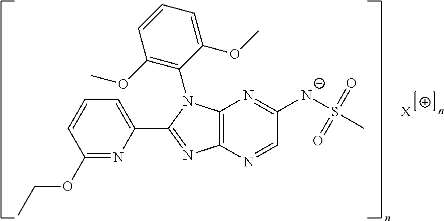

wherein: X is sodium and n is 1; X is potassium and n is 1, X is calcium and n is 2, or X is magnesium and n is 2.

In one embodiment, provided is N-(1-(2,6-dimethoxyphenyl)-2-(6-ethoxypyridin-2-yl)-1H-imidazo[4,5-b]pyrazin-6-yl)methanesulfonamide sodium salt (Compound I sodium salt), or solvate thereof. In one embodiment, provided is N-(1-(2,6-dimethoxyphenyl)-2-(6-ethoxypyridin-2-yl)-1H-imidazo[4,5-b]pyrazin-6-yl)methanesulfonamide potassium salt (Compound I potassium salt), or solvate thereof. In one embodiment, provided is N-(1-(2,6-dimethoxyphenyl)-2-(6-ethoxypyridin-2-yl)-1H-imidazo[4,5-b]pyrazin-6-yl)methanesulfonamide calcium salt (Compound I calcium salt), or solvate thereof. In one embodiment, provided is N-(1-(2,6-dimethoxyphenyl)-2-(6-ethoxypyridin-2-yl)-1H-imidazo[4,5-b]pyrazin-6-yl)methanesulfonamide magnesium salt (Compound I magnesium salt), or solvate thereof.

In one embodiment, provided is a crystalline salt form of N-(1-(2,6-dimethoxyphenyl)-2-(6-ethoxypyridin-2-yl)-1H-imidazo[4,5-b]pyrazin-6-yl)methanesulfonamide (Compound I), or solvate thereof, having the Formula:

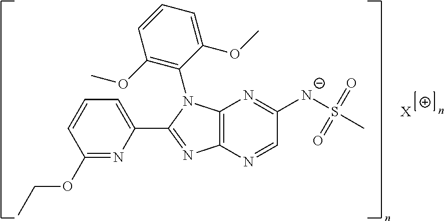

wherein: X is sodium and n is 1; X is potassium and n is 1, X is calcium and n is 2, or X is magnesium and n is 2.

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide sodium salt (Compound I sodium salt), or solvate thereof. In one embodiment, provided is crystalline N-(1-(2,6-dimethoxyphenyl)-2-(6-ethoxypyridin-2-yl)-1H-imidazo[4,5-b]pyrazin-6-yl)methanesulfonamide potassium salt (Compound I potassium salt), or solvate thereof. In one embodiment, provided is crystalline N-(1-(2,6-dimethoxyphenyl)-2-(6-ethoxypyridin-2-yl)-1H-imidazo[4,5-b]pyrazin-6-yl)methanesulfonamide calcium salt (Compound I calcium salt), or solvate thereof. In one embodiment, provided is crystalline N-(1-(2,6-dimethoxyphenyl)-2-(6-ethoxypyridin-2-yl)-1H-imidazo[4,5-b]pyrazin-6-yl)methanesulfonamide magnesium salt (Compound I magnesium salt), or solvate thereof.

In one embodiment, provided is a N-(1-(2,6-dimethoxyphenyl)-2-(6-ethoxypyridin-2-yl)-1H-imidazo[4,5-b]pyrazin-6-yl)methanesulfonamide choline salt (Compound I choline salt), or solvate thereof, having the formula:

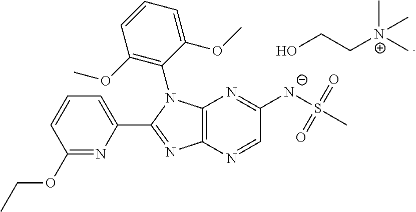

In some embodiments, the Compound I choline salt is a hydrate. In some embodiments, the Compound I choline salt is a mono-choline salt (Compound I free acid:choline=about 1:1). In some embodiments, the Compound I choline salt is a mono-choline salt (Compound I free acid:choline=1:0.9-1.1). In some embodiments, the Compound I choline salt is a mono-choline salt (Compound I free acid:choline=1:0.9). In some embodiments, the Compound I choline salt is a mono-choline salt (Compound I free acid:choline=1:1.1). In some embodiments, the Compound I choline salt is a mono-choline salt (Compound I free acid:choline=1:1) hydrate. In some embodiments, the hydrate is a non-stoichiometric hydrate (channel hydrate).

In some embodiments, the Compound I choline salt, or solvate thereof, is crystalline. In one embodiment, provided is crystalline N-(1-(2,6-dimethoxyphenyl)-2-(6-ethoxypyridin-2-yl)-1H-imidazo[4,5-b]pyrazin-6-yl)methanesulfonamide choline salt (Compound I choline salt), or solvate thereof. In one embodiment, provided is crystalline N-(1-(2,6-dimethoxyphenyl)-2-(6-ethoxypyridin-2-yl)-1H-imidazo[4,5-b]pyrazin-6-yl)methanesulfonamide choline salt (Compound I choline salt) hydrate. In some embodiments, the hydrate is a non-stoichiometric hydrate (channel hydrate).

### Forms of Compound I

### Compound I Choline Salt Form A

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide choline salt Form A (Compound I choline salt Form A). In one embodiment, provided is crystalline N-(1-(2,6-dimethoxyphenyl)-2-(6-ethoxypyridin-2-yl)-1H-imidazo[4,5-b]pyrazin-6-yl)methanesulfonamide choline salt Form A (Compound I choline salt Form A), characterized by an X-ray powder diffractogram comprising the following peaks expressed in ±0.2 degrees 2-theta selected from: 12.9, 14.6, and 18.1, as determined on a diffractometer using Cu-Kα radiation.

In some embodiments, Compound I choline salt Form A is further characterized by an X-ray powder diffractogram comprising one or more additional peaks expressed in ±0.2 degrees 2-theta selected from: 9.9, 16.7, 17.5, 19.8, 22.7, and 23.1, as determined on a diffractometer using Cu-Kα radiation.

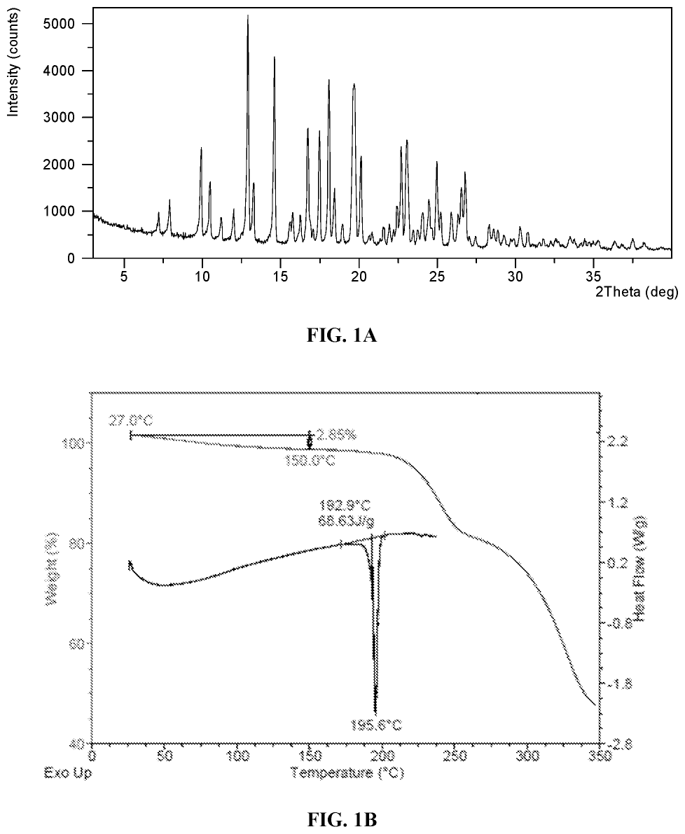

In some embodiments, Compound I choline salt Form A is further characterized by an X-ray powder diffractogram as substantially shown in FIG. 1A.

In some embodiments, Compound I choline salt Form A is further characterized by a DSC comprising an endotherm at about 194-200° C. (peak), or about 194-196° C. (peak), or about 195-196° C. (peak). In some embodiments, Compound I choline salt Form A is further characterized by a DSC comprising an endotherm at about 194° C., or 194.2° C., or about 195° C., or 195.1° C., or 195.6° C., or about 196° C., or about 197° C., or about 200° C. (peak). In some embodiments, Compound I choline salt Form A is further characterized by a DSC comprising a broad endotherm at 104.0° C. (peak) and a peak at 199.7° C. (peak). In some embodiments, Compound I choline salt Form A is further characterized by a DSC as substantially shown in FIG. 1B.

In some embodiments, crystalline Compound I choline salt Form A is prepared via slurry of Compound I free acid Form A and equimolar choline in acetone at room temperature for 4 days.

In some embodiments, Compound I choline salt Form A is further characterized by TGA showing a weight loss of 1.9% up to 150° C. In some embodiments, Compound I choline salt Form A is further characterized by TGA showing a weight loss of 3.3% up to 150° C.

In some embodiments, the molar ratio of choline/Compound I free acid in Compound I choline salt Form A is about 1.0. In some embodiments, the molar ratio of choline/Compound I free acid in Compound I choline salt Form A is 0.9. In some embodiments, the molar ratio of choline/Compound I free acid in Compound I choline salt Form A is 1.1-0.9.

In some embodiments, Compound I choline salt Form A is a mono-choline salt (Compound I free acid:choline=about 1:1). In some embodiments, Compound I choline salt Form A is a mono-choline salt (Compound I free acid:choline=1:0.9-1.1).

In some embodiments, the molar ratio of acetone/Compound I in Compound I choline salt Form A is 0.04 (0.4 wt %). In some embodiments, the molar ratio of acetone/Compound I in Compound I choline salt Form A is 0.01 (0.1 wt %). In some embodiments, the molar ratio of acetone/Compound I in Compound I choline salt Form A is less than 0.01 (0.1 wt %).

In some embodiments, Compound I choline salt Form A is further characterized as a single crystal. Accordingly, in one embodiment, provided is crystalline N-(1-(2,6-dimethoxyphenyl)-2-(6-ethoxypyridin-2-yl)-1H-imidazo[4,5-b]pyrazin-6-yl)methanesulfonamide choline salt Form A (Compound I choline salt Form A), having unit cell parameters: α=8.4179(3) Å, b=22.3688(7) Å, c=14.7788(6) Å, α=90°, β=92.803(3)°, γ=90°, V=2779.49(17) ∈³.

### Compound I Choline Salt Form B

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide choline salt Form B (Compound I choline salt Form B). In one embodiment, provided is crystalline N-(1-(2,6-dimethoxyphenyl)-2-(6-ethoxypyridin-2-yl)-1H-imidazo[4,5-b]pyrazin-6-yl)methanesulfonamide choline salt Form B (Compound I choline salt Form B), characterized by an X-ray powder diffractogram comprising the following peaks expressed in ±0.2 degrees 2-theta selected from: 11.0, 14.5, and 19.2, as determined on a diffractometer using Cu-Kα radiation.

In some embodiments, Compound I choline salt Form B is further characterized by an X-ray powder diffractogram comprising one or more additional peaks expressed in ±0.2 degrees 2-theta selected from: 18.4, 22.1, and 24.4, as determined on a diffractometer using Cu-Kα radiation. In some embodiments, Compound I choline salt Form B is further characterized by an X-ray powder diffractogram comprising one or more additional peaks expressed in ±0.2 degrees 2-theta selected from: 18.7, 19.8, and 21.6, as determined on a diffractometer using Cu-Kα radiation. In some embodiments, Compound I choline salt Form B is further characterized by an X-ray powder diffractogram comprising one or more additional peaks expressed in ±0.2 degrees 2-theta selected from: 18.4, 18.7, 19.8, 21.6, 22.1, and 24.4, as determined on a diffractometer using Cu-Kα radiation.

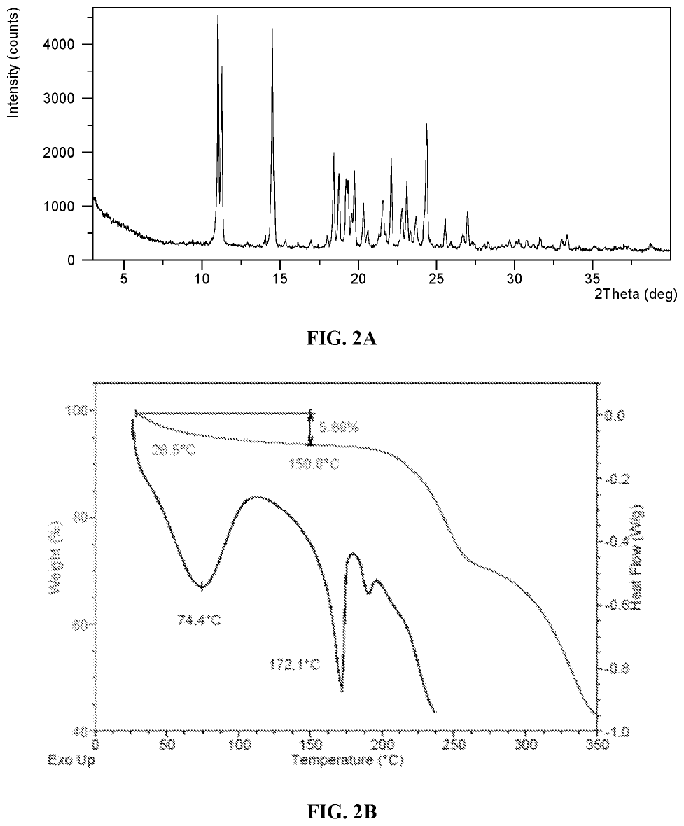

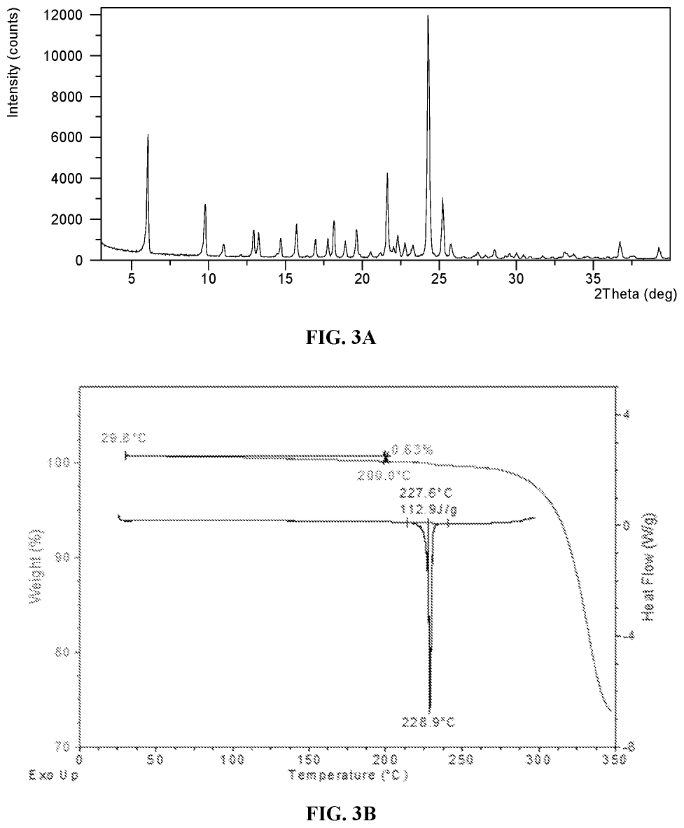

In some embodiments, Compound I choline salt Form B is further characterized by an X-ray powder diffractogram as substantially shown in FIG. 2A.

In some embodiments, Compound I choline salt Form B is further characterized by a DSC comprising a peak at 74.4° C. (peak) and 172.1° C. (peak). In some embodiments, Compound I choline salt Form B is further characterized by a DSC as substantially shown in FIG. 2B.

In some embodiments, Compound I choline salt Form B is obtained via slurry free acid Form A and equimolar choline in acetone and 1,4-dioxane, respectively, at RT for 4 days.

In some embodiments, Compound I choline salt Form B is further characterized by TGA showing a weight loss of 5.9% up to 150° C.

In some embodiments, the molar ratio of choline/Compound I free acid in Compound I choline salt Form B is 1.2. In some embodiments, the molar ratio of 1,4-dioxane/Compound I free acid in Compound I choline salt Form B is 0.7 (6.2 wt %).

### Compound I Free Acid Form B

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide Form B (Compound I free acid Form B). In one embodiment, provided is crystalline N-(1-(2,6-dimethoxyphenyl)-2-(6-ethoxypyridin-2-yl)-1H-imidazo[4,5-b]pyrazin-6-yl)methanesulfonamide Form B (Compound I free acid Form B), characterized by an X-ray powder diffractogram comprising the following peaks expressed in ±0.2 degrees 2-theta selected from: 9.5, 13.8, and 15.2, as determined on a diffractometer using Cu-Kα radiation.

In some embodiments, Compound I free acid Form B is further characterized by an X-ray powder diffractogram comprising one or more additional peaks expressed in ±0.2 degrees 2-theta selected from: 14.2, 17.1, 19.6, 26.0, 26.9, and 27.9°2θ±0.2°2θ, as determined on a diffractometer using Cu-Kα radiation.

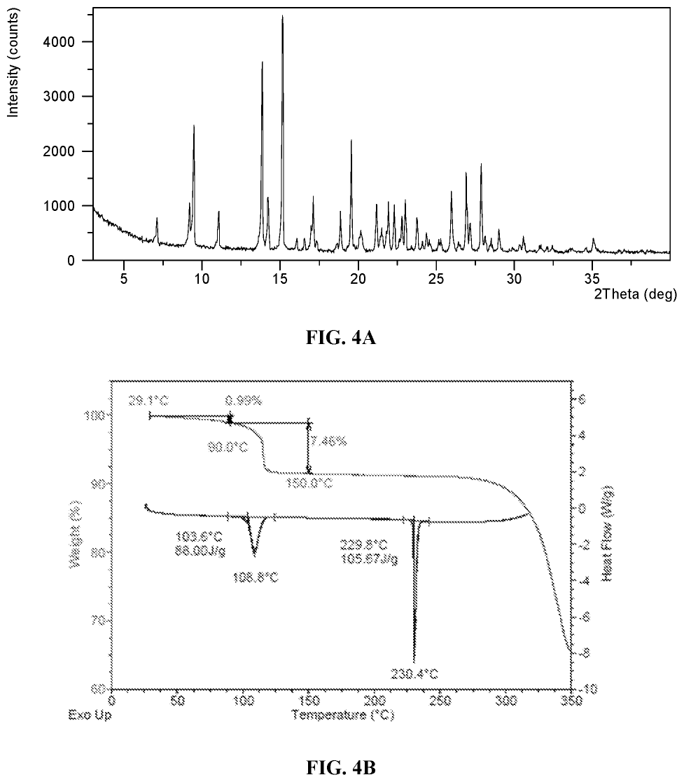

In some embodiments, Compound I free acid Form B is further characterized by an X-ray powder diffractogram as substantially shown in FIG. 4A.

In some embodiments, Compound I free acid Form B is further characterized by a DSC comprising two endotherms at 108.8° C. (peak) and 230.4° C. (peak). In some embodiments, crystalline Compound I free acid Form B is further characterized by a DSC as substantially shown in FIG. 4B.

In some embodiments, Compound I free acid Form B is obtained via slurry of Compound I free acid Form A in ACN:H₂O (9:1, v/v) at room temperature for 4 days.

In some embodiments, Compound I free acid Form B is further characterized by TGA showing a weight loss of 8.5% up to 150° C.

In some embodiments, the molar ratio of ACN/Compound I free acid in Compound I free acid Form B is 0.8 (6.1 wt %).

### Compound I Sodium Salt Form A

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide sodium salt Form A (Compound I sodium salt Form A).

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide sodium salt Form A (Compound I sodium salt Form A), characterized by an X-ray powder diffractogram comprising the following peaks expressed in ±0.2 degrees 2-theta selected from: 8.9, 15.5, and 26.3, as determined on a diffractometer using Cu-Kα radiation.

In some embodiments, Compound I sodium salt Form A is further characterized by an X-ray powder diffractogram comprising one or more additional peaks expressed in ±0.2 degrees 2-theta selected from: 12.2, 12.6, and 15.3, as determined on a diffractometer using Cu-Kα radiation. In some embodiments, Compound I sodium salt Form A is further characterized by an X-ray powder diffractogram comprising one or more additional peaks expressed in ±0.2 degrees 2-theta selected from: 7.8, 23.1, and 25.5, as determined on a diffractometer using Cu-Kα radiation. In some embodiments, Compound I sodium salt Form A is further characterized by an X-ray powder diffractogram comprising one or more additional peaks expressed in ±0.2 degrees 2-theta selected from: 7.8, 12.2, 12.6, 15.3, 23.1, and 25.5, as determined on a diffractometer using Cu-Kα radiation.

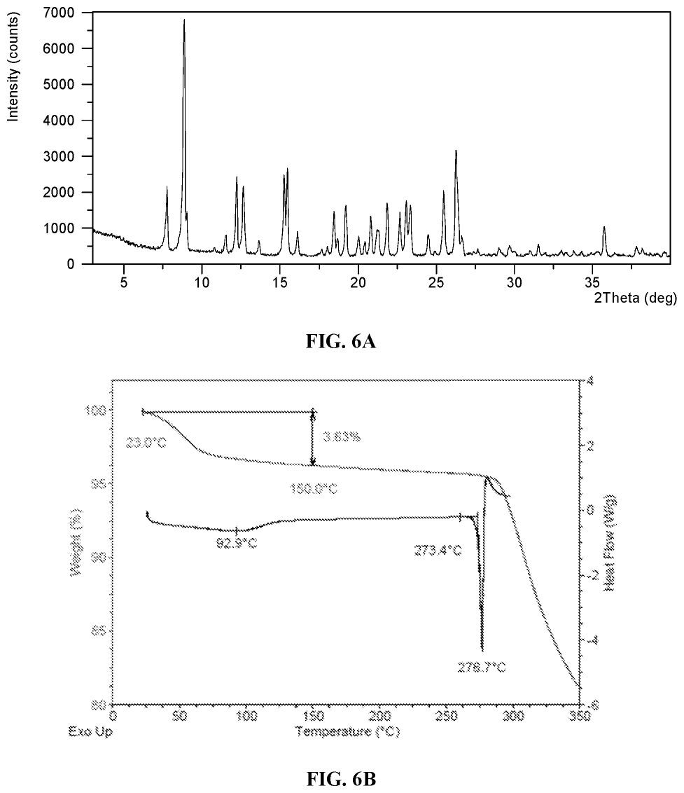

In some embodiments, Compound I sodium salt Form A is characterized by an X-ray powder diffractogram as substantially shown in FIG. 6A.

In some embodiments, Compound I sodium salt Form A is further characterized by a DSC comprising two endotherms at 92.9 and 276.7° C. (peak). In some embodiments, crystalline Compound I sodium salt Form A is further characterized by a DSC as substantially shown in FIG. 6B.

In some embodiments, Compound I sodium salt Form A is obtained via slurry Compound I free acid Form A and equimolar NaOH in acetone at room temperature for 4 days.

In some embodiments, Compound I sodium salt Form A is further characterized by TGA showing a weight loss of 3.6% up to 150° C.

In some embodiments, Compound I sodium salt Form A is obtained via slurry Compound I free acid Form A and equimolar NaOH in acetone at room temperature for 4 days.

In some embodiments, crystalline Compound I sodium salt Form A is a solvate. In some embodiments, crystalline Compound I sodium salt Form A is an acetone solvate. In some embodiments, the Compound I sodium salt Form A acetone solvate has a molar ratio of acetone/Compound I of 0.01 (0.1 wt %). In some embodiments, the molar ratio of crystalline Compound I sodium salt Form A is 1:1 sodium:Compound I free acid.

### Compound I Potassium Salt Form A

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide potassium salt Form A (Compound I potassium salt Form A).

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide potassium salt Form A (Compound I potassium salt Form A), characterized by an X-ray powder diffractogram comprising the following peaks expressed in ±0.2 degrees 2-theta selected from: 12.3, 14.8, and 26.3, as determined on a diffractometer using Cu-Kα radiation.

In some embodiments, Compound I potassium salt Form A is further characterized by an X-ray powder diffractogram comprising one or more additional peaks expressed in ±0.2 degrees 2-theta selected from: 8.7, 22.3, and 25.9, as determined on a diffractometer using Cu-Kα radiation. In some embodiments, Compound I potassium salt Form A is further characterized by an X-ray powder diffractogram comprising one or more additional peaks expressed in ±0.2 degrees 2-theta selected from: 7.9, 15.7, and 21.3, as determined on a diffractometer using Cu-Kα radiation. In some embodiments, Compound I potassium salt Form A is further characterized by an X-ray powder diffractogram comprising one or more additional peaks expressed in ±0.2 degrees 2-theta selected from: 7.9, 8.7, 15.7, 21.3, 22.3, and 25.9, as determined on a diffractometer using Cu-Kα radiation.

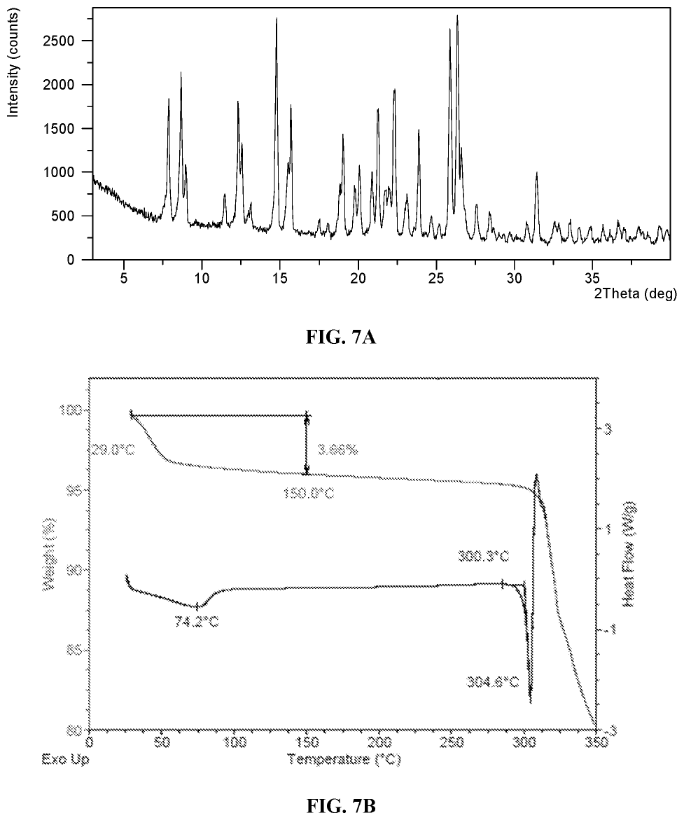

In some embodiments, Compound I potassium salt Form A is characterized by an X-ray powder diffractogram as substantially shown in FIG. 7A.

In some embodiments, Compound I potassium salt Form A is further characterized by a DSC comprising two endotherms at 74.2° C. and 304.6° C. (peak). In some embodiments, Compound I potassium salt Form A is further characterized by a DSC as substantially shown in FIG. 7B.

In some embodiments, Compound I potassium salt Form A is provided via slurry Compound I free acid Form A and equimolar KOH in acetone at room temperature for 4 days.

In some embodiments, Compound I potassium salt Form A is further characterized by TGA showing a weight loss of 3.7% up to 150° C.

In some embodiments, the Compound I potassium salt Form A acetone solvate has a molar ratio of acetone/Compound I of 0.03 (0.3 wt %). In some embodiments, the Compound I potassium salt Form A is 1:1 potassium:Compound I free acid.

### Compound I Potassium Salt Form B

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide potassium salt Form B (Compound I potassium salt Form B).

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide potassium salt Form B (Compound I potassium salt Form B), characterized by an X-ray powder diffractogram comprising the following peaks expressed in ±0.2 degrees 2-theta selected from: 6.8, 7.8, and 8.6, as determined on a diffractometer using Cu-Kα radiation.

In some embodiments, Compound I potassium salt Form B is further characterized by an X-ray powder diffractogram comprising one or more additional peaks expressed in ±0.2 degrees 2-theta selected from: 11.8, 14.7, and 22.3, as determined on a diffractometer using Cu-Kα radiation. In some embodiments, Compound I potassium salt Form B is further characterized by an X-ray powder diffractogram comprising one or more additional peaks expressed in ±0.2 degrees 2-theta selected from: 21.2, 25.8, and 26.3, as determined on a diffractometer using Cu-Kα radiation. In some embodiments, Compound I potassium salt Form B is further characterized by an X-ray powder diffractogram comprising one or more additional peaks expressed in ±0.2 degrees 2-theta selected from: 11.8, 14.7, 21.2, 22.3, 25.8, and 26.3, as determined on a diffractometer using Cu-Kα radiation.

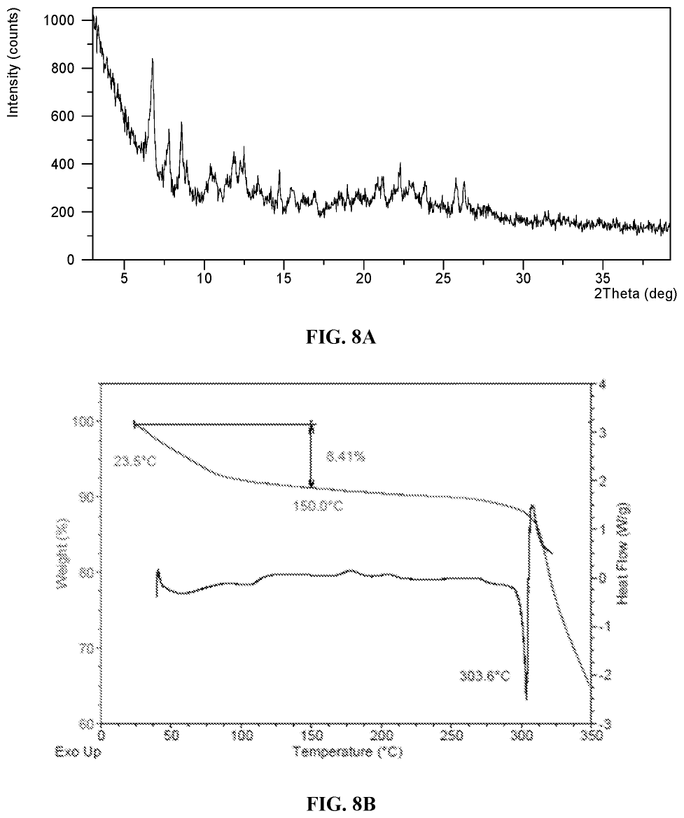

In some embodiments, Compound I potassium salt Form B is characterized by an X-ray powder diffractogram as substantially shown in FIG. 8A.

In some embodiments, Compound I potassium salt Form B is further characterized by a DSC comprising an endotherm at 303.6° C. (peak). In some embodiments, Compound I potassium salt Form B is further characterized by a DSC as substantially shown in FIG. 8B.

In some embodiments, Compound I potassium salt Form B is provided via a slurry of Compound I free acid Form A and equimolar KOH in MeOH at −20° C. for 4 days.

In some embodiments, Compound I potassium salt Form B is further characterized by TGA showing a weight loss of 8.4% up to 150° C.

In some embodiments, Compound I potassium salt Form B is an ansolvate. In some embodiments, Compound I potassium salt Form B is 1:1 potassium:Compound I free acid.

### Compound I Calcium Salt Form A

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide calcium salt Form A (Compound I calcium salt Form A).

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide calcium salt Form A (Compound I calcium salt Form A), characterized by an X-ray powder diffractogram comprising the following peaks expressed in ±0.2 degrees 2-theta selected from: 12.1, 16.6, and 25.6, as determined on a diffractometer using Cu-Kα radiation.

In some embodiments, Compound I calcium salt Form A is further characterized by an X-ray powder diffractogram comprising one or more additional peaks expressed in ±0.2 degrees 2-theta selected from: 20.4, 22.4, and 23.9, as determined on a diffractometer using Cu-Kα radiation. In some embodiments, Compound I calcium salt Form A is further characterized by an X-ray powder diffractogram comprising one or more additional peaks expressed in ±0.2 degrees 2-theta selected from: 6.7, 13.3, and 24.4, as determined on a diffractometer using Cu-Kα radiation. In some embodiments, Compound I calcium salt Form A is further characterized by an X-ray powder diffractogram comprising one or more additional peaks expressed in ±0.2 degrees 2-theta selected from: 6.7, 13.3, 20.4, 22.4, 23.9, and 24.4, as determined on a diffractometer using Cu-Kα radiation.

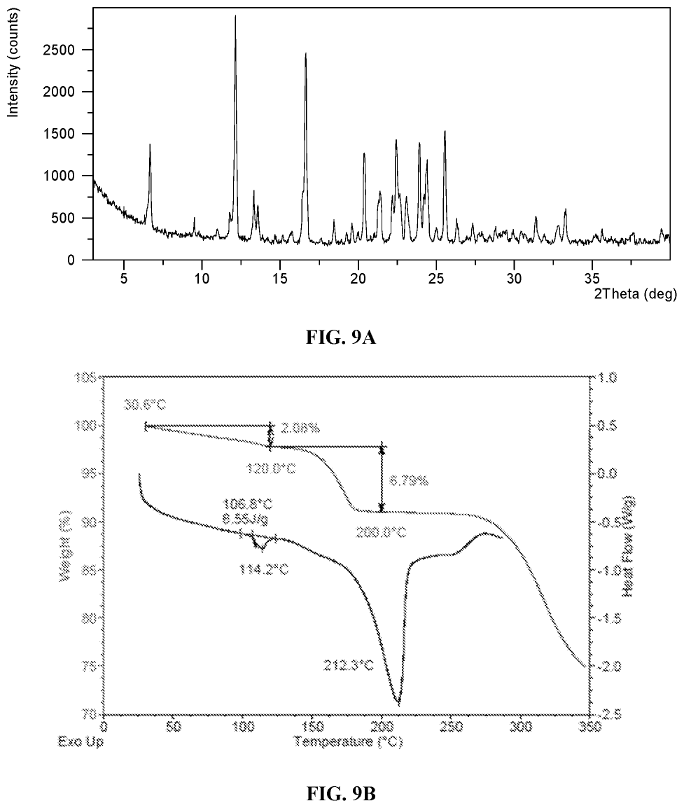

In some embodiments, Compound I calcium salt Form A is characterized by an X-ray powder diffractogram as substantially shown in FIG. 9A.

In some embodiments, Compound I calcium salt Form A is further characterized by a DSC comprising two endotherms at 114.2 and 212.3° C. In some embodiments, Compound I calcium salt Form A is further characterized by a DSC as substantially shown in FIG. 9B.

In some embodiments, Compound I calcium salt Form A is obtained via slurry of Compound I free acid Form A and equimolar Ca(OH)₂ in ACN:H₂O (9:1, v/v) at room temperature for 4 days.

In some embodiments, Compound I calcium salt Form A is obtained via slurry of Compound I free acid Form A and Ca(OH)₂ (molar ratio of 0.5, base/acid) in ACN:H₂O (9:1, v/v) at room temperature for 1 day.

In some embodiments, Compound I calcium salt Form A is further characterized by TGA showing a two-step weight loss of 2.10% up to 120° C. and 6.8% from 120° C. to 200° C.

In some embodiments, the Compound I calcium salt Form A has a molar ratio of ACN/Compound I of 0.07 (0.6 wt %). In some embodiments, the Compound I calcium salt Form A is 1:0.5 calcium:Compound I free acid.

### Compound I Magnesium Salt Form A

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide magnesium salt Form A (Compound I magnesium salt Form A).

In one embodiment, provided is crystalline N-(1-(2,6-dimethoxyphenyl)-2-(6-ethoxypyridin-2-yl)-1H-imidazo[4,5-b]pyrazin-6-yl)methanesulfonamide magnesium salt Form A (Compound I choline salt Form B), characterized by an X-ray powder diffractogram comprising the following peaks expressed in ±0.2 degrees 2-theta selected from: 13.7, 13.9, and 14.8, as determined on a diffractometer using Cu-Kα radiation.

In some embodiments, Compound I magnesium salt Form A is further characterized by an X-ray powder diffractogram comprising one or more additional peaks expressed in ±0.2 degrees 2-theta selected from: 19.0, 25.3, and 26.0, as determined on a diffractometer using Cu-Kα radiation. In some embodiments, Compound I magnesium salt Form A is further characterized by an X-ray powder diffractogram comprising one or more additional peaks expressed in ±0.2 degrees 2-theta selected from: 22.0, 23.1, and 23.3, as determined on a diffractometer using Cu-Kα radiation. In some embodiments, Compound I magnesium salt Form A is further characterized by an X-ray powder diffractogram comprising one or more additional peaks expressed in 0.2 degrees 2-theta selected from: 19.0, 22.0, 23.1, 23.3, 25.3, and 26.0, as determined on a diffractometer using Cu-Kα radiation.

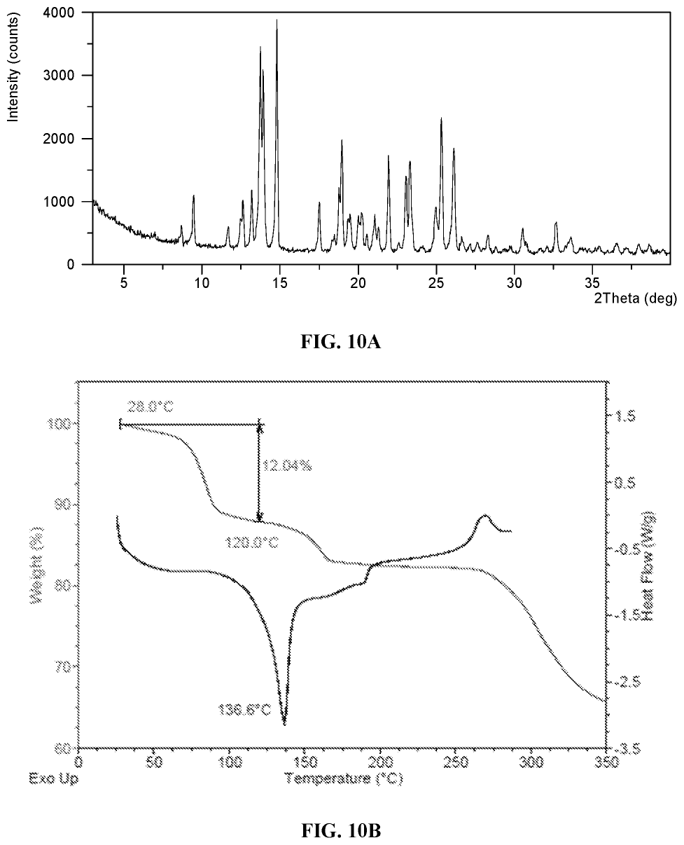

In some embodiments, Compound I magnesium salt Form A is characterized by an X-ray powder diffractogram as substantially shown in FIG. 10A.

In some embodiments, Compound I magnesium salt Form A is further characterized by a DSC comprising an endotherm at 136.6° C. (peak). In some embodiments, Compound I magnesium salt Form A is further characterized by a DSC as substantially shown in FIG. 10B.

In some embodiments, Compound I magnesium salt Form A is obtained via slurry of Compound I free acid Form A and equimolar Mg(OH)₂ in ACN:H₂O (9:1, v/v) at room temperature for 4 days.

In some embodiments, Compound I magnesium salt Form A is obtained via slurry Compound I free acid Form A and Mg(OH)₂ (molar ratio of 0.5, Mg(OH)₂/Compound I free acid) in ACN:H₂O (9:1, v/v) at room temperature for 2 days.

In some embodiments, Compound I magnesium salt Form A is further characterized by TGA showing a weight loss of 12.0% up to 120° C.

In some embodiments, Compound I magnesium salt Form A has a molar ratio of ACN/Compound I of 0.71 (5.7 wt %). In some embodiments, the Compound I magnesium salt Form A is 1:0.5 magnesium:Compound I free acid.

### Compositions

In some embodiments, provided is a composition comprising a salt or crystalline Form of N-(1-(2,6-dimethoxyphenyl)-2-(6-ethoxypyridin-2-yl)-1H-imidazo[4,5-b]pyrazin-6-yl)methanesulfonamide (Compound I), or salt or solvate thereof, as described herein.

In one embodiment, provided is a composition comprising a salt or crystalline Form of N-(1-(2,6-dimethoxyphenyl)-2-(6-ethoxypyridin-2-yl)-1H-imidazo[4,5-b]pyrazin-6-yl)methanesulfonamide (Compound I), or salt or solvate thereof, wherein at least 50% to 99% (e.g., at least 50%, at least 55%, at least 60%, at least 65%, at least 70%, at least 75%, at least 80%, at least 85%, at least 90%, at least 95%, at least 96%, or at least 99%) of Compound I present in the composition is in the designated salt, crystalline Form, or crystalline salt Form.

In one embodiment, provided is a composition comprising N-(1-(2,6-dimethoxyphenyl)-2-(6-ethoxypyridin-2-yl)-1H-imidazo[4,5-b]pyrazin-6-yl)methanesulfonamide choline salt (Compound I choline salt), or solvate thereof, wherein at least 50% to 99% (e.g., at least 50%, at least 55%, at least 60%, at least 65%, at least 70%, at least 75%, at least 80%, at least 85%, at least 90%, at least 95%, at least 96%, or at least 99%) of Compound I present in the composition is Compound I choline salt.

In one embodiment, provided is a composition comprising crystalline N-(1-(2,6-dimethoxyphenyl)-2-(6-ethoxypyridin-2-yl)-1H-imidazo[4,5-b]pyrazin-6-yl)methanesulfonamide choline salt Form A (Compound I choline salt Form A), or solvate thereof, wherein at least 50% to 99% (e.g., at least 50%, at least 55%, at least 60%, at least 65%, at least 70%, at least 75%, at least 80%, at least 85%, at least 90%, at least 95%, at least 96%, or at least 99%) of Compound I present in the composition is Compound I choline salt Form A.

In one embodiment, provided is a composition comprising crystalline N-(1-(2,6-dimethoxyphenyl)-2-(6-ethoxypyridin-2-yl)-1H-imidazo[4,5-b]pyrazin-6-yl)methanesulfonamide choline salt Form B (Compound I choline salt Form B), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I choline salt Form B.

In one embodiment, provided is a composition comprising crystalline N-(1-(2,6-dimethoxyphenyl)-2-(6-ethoxypyridin-2-yl)-1H-imidazo[4,5-b]pyrazin-6-yl)methanesulfonamide choline salt Form B (Compound I free acid Form B), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I free acid Form B.

In one embodiment, provided is a composition comprising crystalline N-(1-(2,6-dimethoxyphenyl)-2-(6-ethoxypyridin-2-yl)-1H-imidazo[4,5-b]pyrazin-6-yl)methanesulfonamide sodium salt Form A (Compound I sodium salt Form A), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I sodium salt Form A.

In one embodiment, provided is a composition comprising crystalline N-(1-(2,6-dimethoxyphenyl)-2-(6-ethoxypyridin-2-yl)-1H-imidazo[4,5-b]pyrazin-6-yl)methanesulfonamide potassium salt Form A (Compound I potassium salt Form A), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I potassium salt Form A.

In one embodiment, provided is a composition comprising crystalline N-(1-(2,6-dimethoxyphenyl)-2-(6-ethoxypyridin-2-yl)-1H-imidazo[4,5-b]pyrazin-6-yl)methanesulfonamide potassium salt Form B (Compound I potassium salt Form B), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I potassium salt Form B.

In one embodiment, provided is a composition comprising crystalline N-(1-(2,6-dimethoxyphenyl)-2-(6-ethoxypyridin-2-yl)-1H-imidazo[4,5-b]pyrazin-6-yl)methanesulfonamide calcium salt Form A (Compound I calcium salt Form A), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I calcium salt Form A.

In one embodiment, provided is a composition comprising crystalline N-(1-(2,6-dimethoxyphenyl)-2-(6-ethoxypyridin-2-yl)-1H-imidazo[4,5-b]pyrazin-6-yl)methanesulfonamide magnesium salt Form A (Compound I magnesium salt Form A), or solvate thereof, wherein at least 50% to 99% (e.g., at least 50%, at least 55%, at least 60%, at least 65%, at least 70%, at least 75%, at least 80%, at least 85%, at least 90%, at least 95%, or at least 99%) of Compound I present in the composition is Compound I magnesium salt Form A.

In some embodiments, the composition is a pharmaceutical composition which further comprises a pharmaceutically acceptable excipient.

In some embodiments, provided is a process for preparing crystalline N-(1-(2,6-dimethoxyphenyl)-2-(6-ethoxypyridin-2-yl)-1H-imidazo[4,5-b]pyrazin-6-yl)methanesulfonamide choline salt (Compound I choline salt) comprising contacting Compound I, or a salt thereof, with choline in a solvent for a time sufficient to provide a crystalline Compound I choline salt.

In some embodiments, the solvent is acetone. In some embodiments, the solvent is 1,4-dioxane.

In some embodiments, the contacting comprises adding an equimolar amount of choline to Compound I. In some embodiments, the contacting comprises adding an equimolar amount of choline to Compound I at a temperature of about 0° C. to about 50° C. In some embodiments, the contacting comprises adding an equimolar amount of choline to Compound I at a temperature of about 20° C. to about 30° C.

In some embodiments, the process further comprises, following said contacting step, isolating the crystalline Compound I choline salt.

In some embodiments, the isolating comprises the steps of filtering, washing, and drying the crystalline Compound I choline salt.

In some embodiments, provided is a process for preparing crystalline Compound I choline salt wherein at least about 95% of the crystalline Compound I choline salt is Form A.

In some embodiments, provided is a process for preparing crystalline Compound I choline salt wherein at least about 95% of the crystalline Compound I choline salt is Form B.

In some embodiments, provided is a process for preparing crystalline N-(1-(2,6-dimethoxyphenyl)-2-(6-ethoxypyridin-2-yl)-1H-imidazo[4,5-b]pyrazin-6-yl)methanesulfonamide sodium salt (Compound I sodium salt) comprising contacting Compound I, or a salt thereof, with sodium hydroxide in a solvent for a time sufficient to provide a crystalline Compound I sodium salt.

In some embodiments, the solvent is acetone.

In some embodiments, the contacting comprises adding an equimolar amount of sodium hydroxide to Compound I. In some embodiments, the contacting comprises adding an equimolar amount of sodium hydroxide to Compound I at a temperature of about 0° C. to about 50° C. In some embodiments, the contacting comprises adding an equimolar amount of sodium hydroxide to Compound I at a temperature of about 20° C. to about 30° C.

In some embodiments, the process further comprises, following said contacting step, isolating the crystalline Compound I sodium salt.

In some embodiments, the isolating comprises the steps of filtering, washing, and drying the crystalline Compound I sodium salt.

In some embodiments, provided is a process for preparing crystalline Compound I sodium salt wherein at least about 95% of the crystalline Compound I sodium salt is Form A.

In embodiments, provided is a process for preparing crystalline N-(1-(2,6-dimethoxyphenyl)-2-(6-ethoxypyridin-2-yl)-1H-imidazo[4,5-b]pyrazin-6-yl)methanesulfonamide potassium salt (Compound I potassium salt) comprising contacting Compound I, or a salt thereof, with potassium hydroxide in a solvent for a time sufficient to provide a crystalline Compound I potassium salt.

In some embodiments, the solvent is acetone.

In some embodiments, the contacting comprises adding an equimolar amount of potassium hydroxide to Compound I. In some embodiments, the contacting comprises adding an equimolar amount of potassium hydroxide to Compound I at a temperature of about 0° C. to about 50° C. In some embodiments, the contacting comprises adding an equimolar amount of potassium hydroxide to Compound I at a temperature of about 20° C. to about 30° C.

In some embodiments, the process further comprises, following said contacting step, isolating the crystalline Compound I potassium salt.

In some embodiments, the isolating comprises the steps of filtering, washing, and drying the crystalline Compound I potassium salt.

In some embodiments, provided is a process for preparing crystalline Compound I potassium salt wherein at least about 95% of the crystalline Compound I potassium salt is Form A.

### Pharmaceutical Compositions and Administration

In some embodiments, a chemical entity (e.g., a salt or crystalline Form of N-(1-(2,6-dimethoxyphenyl)-2-(6-ethoxypyridin-2-yl)-1H-imidazo[4,5-b]pyrazin-6-yl)methanesulfonamide (Compound I), or salt or solvate thereof, as described herein) that modulates (e.g., agonizes) the APJ receptor is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In general, the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules, sterility is not required. The USP/NF standard is usually sufficient.

Ocular compositions can include, without limitation, one or more of any of the following: viscogens (e.g., carboxymethylcellulose, glycerin, polyvinylpyrrolidone, polyethylene glycol); stabilizers (e.g., pluronic (triblock copolymers), cyclodextrins); preservatives (e.g., benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treating and the particular compound being employed. Proper dosage for a particular situation can be determined by one skilled in the medical arts. In some cases, the total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

This disclosure features methods for treating a subject (e.g., a human) having a disease, disorder, or condition in which a decrease in APJ receptor activity (e.g., repressed or impaired APJ receptor signaling; e.g., repressed or impaired apelin-APJ receptor signaling) or downregulation of endogenous apelin contributes to the pathology and/or symptoms and/or progression of the disease, disorder, or condition. In certain embodiments, the methods described herein can include or further include treating one or more conditions associated, co-morbid or sequela with any one or more of the conditions described herein.

In some embodiments, the method further comprises identifying the subject. In some embodiments, identifying comprises determining the level of one or more of the following parameters in the subject: leukotriene B4 level, pulmonary vascular resistance, pulmonary arterial pressure, cardiac index, pulmonary capillary wedge pressure, right atrial pressure, six-minute walk distance, brain natriuretic peptide level, atrial natriuretic peptide, and diffusion of lung capacity.

In certain embodiments, the chemical entities described herein modulate (e.g., decrease) pulmonary vascular resistance, modulate (e.g., decrease) right ventricular afterload, and modulate (e.g., decrease) mean pulmonary artery pressure. In certain embodiments, the chemical entities described herein reduce the risk of right ventricular failure.

In certain embodiments, the chemical entities described herein modulate vascular tone, modulate fluid homeostasis, modulate kidney function, modulate energy metabolism, modulate inflammatory response, and modulate thrombosis.

### Indications

### Pulmonary Hypertension

In some embodiments, the condition, disease or disorder is pulmonary arterial hypertension (PAH). Non-limiting examples of PAH and related conditions include idiopathic PAH, heritable PAH (e.g., BMPR2 mutations and other mutations), drug-induced or toxin-induced PAH, and PAH associated with conditions including but not limited to connective tissue diseases (CTD) (e.g., scleroderma, systemic lupus erythematosus, systemic sclerosis, Hashimoto's thyroiditis, Sjogren's Syndrome, and the antiphospholipid antibody syndrome), HIV infection, portal hypertension, congenital heart disease, and schistosomiasis.

In some embodiments, the PAH is idiopathic.

In other embodiments, the PAH is heritable PAH, toxin or drug-induced PAH; or a PAH associated with one or more of the following: congenital heart disease, connective tissue disorders (e.g., scleroderma, systemic lupus erythematosus, systemic sclerosis, Hashimoto's thyroiditis, Sjogren's Syndrome, and the antiphospholipid antibody syndrome), portal hypertension, BMPR2 mutations, Schistosomiasis, and HIV infection.

In some embodiments, the condition, disease or disorder is pulmonary hypertension other than PAH. Examples of such conditions include, without limitation, pulmonary hypertension due to left heart disease (e.g., left ventricular systolic dysfunction, left ventricular diastolic dysfunction, valvular heart disease, and congenital/acquired left heart inflow/outflow obstruction and congenital cardiomyopathies), pulmonary hypertension due to lung disease and/or hypoxia (e.g., chronic obstructive pulmonary disease, interstitial lung disease, other pulmonary disease with mixed restrictive and obstructive pattern, sleep-disordered breathing, alveolar hypoventilation disorders, chronic exposure to high altitude, developmental lung disease), chronic thromboembolic pulmonary hypertension and other pulmonary artery obstructions (e.g., chronic thromboembolic pulmonary hypertension, other pulmonary artery obstructions), and pulmonary hypertension with unclear multifactorial mechanisms (e.g., hematologic disorders, systemic disorders, metabolic disorders, and others).

### Cardiovascular Conditions, Diseases or Disorders

In some embodiments, the condition, disease or disorder is a cardiovascular condition, disease or disorder. Non-limiting examples of cardiovascular condition, disease or disorder include coronary heart disease, acute coronary syndrome, peripheral vascular disease, angina, stroke, cerebrovascular accidents, transient ischemic attacks, heart failure, cardiomyopathy, myocardial infarction, myocardial remodeling after cardiac surgery, valvular heart disease, hypertension (e.g., systemic hypertension, essential hypertension, pulmonary hypertension, portal hypertension, systolic hypertension), aortic aneurysm (e.g., abdominal aortic aneurysm), atrial fibrillation, arrhythmia, atherosclerosis, Brugada syndrome, ischemic cardiovascular diseases, peripheral arterial disease, preeclampsia, ventricular tachycardia, and cardiac fibrosis.

In some embodiments, the cardiovascular condition, disease or disorder is heart failure. Non-limiting examples of heart failure include chronic heart failure, systolic heart failure, diastolic heart failure, diabetic heart failure, congestive heart failure, heart failure with preserved ejection fraction, heart failure with reduced ejection fraction, left ventricular dysfunction (e.g., left ventricular dysfunction after myocardial infarction), right ventricular dysfunction, cardiac hypertrophy, myocardial remodeling, and acute decompensated heart failure (ADHF).

In some embodiments, the cardiovascular condition, disease or disorder is a condition, disease or disorder with vascular pathology (e.g., with increased vascular permeability and nonfunctional blood vessels). Non-limiting examples of such condition, disease or disorder include vascular hypertrophy, vascular remodeling (e.g., vascular stiffness), atherosclerosis, peripheral arterial occlusive disease (PAOD), restenosis (e.g., angioplastic restenosis), thrombosis and vascular permeability disorders, and ischemia and/or reperfusion damage (e.g., ischemia and/or reperfusion damage of the heart, kidney and retina). In some embodiments, the conditions, disease or disorder is vein related. Non-limiting examples of such condition, disease or disorder include angioma, veinous insufficiency, stasis, or thrombosis.

In some embodiments, the chemical entities described herein can improve cardiac contractility (e.g., cardiac relaxation), ventricular arterial coupling, inotropic function, or luistropic function in a subject suffering from a cardiovascular condition. In some embodiments, the chemical entities described herein can increase ejection fraction in a subject suffering from a cardiovascular condition.

### Metabolic and Homeostatic Dysfunction and Related Conditions, Diseases or Disorders

In some embodiments, the condition, disease or disorder is associated with metabolic dysfunction. Non-limiting examples of such condition, disease or disorder include metabolic dysfunction, obesity, diabetes (e.g., type II diabetes mellitus, gestational diabetes), complications of diabetes (e.g., metabolic syndrome, insulin resistance, organ damages of micro- or macrovascular origins such as macro- and microvaculopathies, diabetic neuropathy, diabetic retinopathy, cardiac autonomic neuropathy), kidney disease (e.g., chronic kidney disease), edema, dyslipidemia, anorexia, hyperphagia, polyphagia, hypercholesterolemia, hyperglyceridemia, hyperlipemia, growth hormone disorder (e.g., gigantism, aromegaly), galactorrhea, and cardiac wasting.

In some embodiments, the condition, disease or disorder is associated with inappropriate vasopressin secretions (SIADH). Non-limiting examples of such condition, disease or disorder include neurogenic diabetes mellitus (e.g. diabetic complications such as diabetic nephropathy, diabetic neuropathy, diabetic retinopathy, etc.), lung cancer, septic shock, and thirst troubles.

In some embodiments, the condition, disease or disorder is associated with systemic inflammation. Non-limiting examples of such condition, disease or disorder include systemic inflammatory response syndrome (SIRs), sepsis (e.g., severe sepsis), and septic shock. In some embodiments, the condition, disease or disorder is associated with sepsis (e.g., a complication, co-morbidity, or sequela of sepsis). Non-limiting examples of conditions, diseases or disorders associated with sepsis include sepsis-induced myocardial dysfunction, sepsis-related inflammatory response (e.g., systemic inflammation), sepsis-related hemodynamic alterations, hypovolemia, sepsis-related organ failures (e.g., multi-organ failure, renal failure), acute kidney injury, vasoplegia, lung injury, inappropriate vasopressin secretions, persistent hypertension related to generalized vasodilation, refractory constrictive responsiveness, huge plasma capillary leak syndrome, coagulation/fibrinolysis imbalance, and metabolic disturbance highlighted by elevated blood-stream lactates. See. e.g., Coquerel et al. Critical Care (2018) 22:10.

In some embodiments, the chemical entities described herein can regulate arginine vasopressin (AVP) or angiotensin receptor.

In some embodiments, the condition, disease or disorder is associated with disturbed body's fluid homeostasis by CNS-dependent and -independent effects. Non-limiting examples of such condition, disease or disorder include renal failure (e.g., acute and chronic renal failure), renal perfusion, renal dysfunction (e.g., polycystic kidney disease), aquaresis, and diuresis.

### Dementia and Related Conditions, Diseases or Disorders

In some embodiments, the condition, disease or disorder is dementia. Non-limiting examples of such condition, disease or disorder include senile dementia, cerebrovascular dementia, dementia due to genealogical denaturation degenerative diseases (e.g. Alzheimer's disease, Parkinson's disease, Pick's disease, Huntington's disease, etc.), dementia resulting from infectious diseases (e.g. delayed virus infections such as Creutzfeldt-Jakob disease), dementia associated with endocrine diseases, metabolic diseases, or poisoning (e.g. hypothyroidism, vitamin B12 deficiency, alcoholism, poisoning caused by various drugs, metals, or organic compounds), dementia caused by tumors (e.g. brain tumor), and dementia due to traumatic diseases (e.g. chronic subdural hematoma), depression, hyperactive child syndrome (microencephalopathy), disturbance of consciousness, anxiety disorder, schizophrenia, and phobia.

### Connective Tissue Disorder

In some embodiments, the condition, disease or disorder is a connective tissue disorder. In certain embodiments, the connective tissue disorder is selected from the group consisting of: scleroderma, systemic lupus erythematosus, systemic sclerosis, Hashimoto's thyroiditis, Sjogren's Syndrome, and the antiphospholipid antibody syndrome. In certain embodiments, the condition, disease or disorder is systemic sclerosis.

### Fibrosis

In some embodiments, the condition, disease or disorder is fibrosis. In certain embodiments, the fibrosis is associated with an organ or tissue selected from the group consisting of: lung, liver, heart, mediastinum, bone marrow, retroperitoneum, skin, intestine, joint, a reproductive organ, and a combination thereof. In certain embodiments, the fibrosis is idiopathic pulmonary fibrosis (IPF). In certain embodiments, the fibrosis is liver fibrosis. In certain embodiments, the fibrosis is associated with non-alcoholic fatty liver disease (NAFLD)

### Other Conditions, Diseases or Disorders

In some embodiments, the condition, disease or disorder is a liver disease. Non-limiting examples of such condition, disease or disorder include alcoholic liver disease, toxicant-induced liver disease, viral induced liver disease, and liver cirrhosis.

In some embodiments, the condition, disease or disorder is a pulmonary disease. Non-limiting examples of such condition, disease or disorder include chronic obstructive pulmonary disease (COPD), asthma, acute respiratory distress syndrome (ARDS), and amyotrophic lateral sclerosis. In some embodiments, the condition, disease or disorder is a retinal disease (e.g., macular degeneration).

In some embodiments, the condition, disease or disorder is HIV infection, HIV neurodegeneration, neurodegenerative disease, cancer (e.g., mammary cancer, lymphocytic leukemia, bladder cancer, ovary cancer, carcinoma of prostate, etc.), asthma, burn injuries (e.g., sun burn), traumatic brain injuries, pancreatitis, Turner's syndrome, neurosis, rheumatoid arthritis, spinal cord injury, immune function, inflammation, spinocerebellar degeneration, bone fracture, wounds, atopic dermatitis, osteoporosis, asthma, epilepsy, and sterility.

### Activating Stem Cells

The chemical entities described herein can also be used to activate stem cells (e.g., cardiac stem cells such as endogenous cardiac stem cells). In some embodiments, the chemical entities described herein can be used in regrowing tissue, assisting functional recovery after transplanting cells (e.g., cells with bone marrow-derived mesenchymal stem cells), increasing cardiac stem cell proliferation (e.g., in patents that have suffered a myocardial infarction), reducing infarct size, promoting cardiac repair, activating stem cells and progenitors in postmyocardial infarction subjects, or reducing reperfusion injury (e.g., during surgeries such as heart bypass surgery or heart transplant procedures).

### Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein. In some embodiments, the compound described herein can be administered in combination with one or more of additional therapeutic agents.

Representative additional therapeutic agents include, but are not limited to, therapeutic agents for PAH, pulmonary hypertension, heart failure (e.g., ADHF, chronic heart failure), hypertension (e.g., systemic hypertension), amyotrophic lateral sclerosis, arrhythmia, asthma, atherosclerosis, atrial fibrillation, Brugada syndrome, burn injuries (e.g., sunburn), cancer, cardiac fibrosis, cardiomyopathy, cerebrovascular accidents, diabetes (e.g., gestational diabetes), septic shock, sepsis, renal failure, dyslipidemia, HIV neurodegeneration, inflammation, ischemic cardiovascular disease, liver disease, metabolic disorder, neurodegenerative disease, obesity, peripheral arterial disease, preeclampsia, restenosis, transient ischemic attacks, traumatic brain injuries, ventricular tachycardia, edema, or immune function.

In some embodiments, the one or more additional therapeutic agents include those useful, e.g., as therapeutics for PAH. Non-limiting examples include prostacyclin analogues (e.g., epoprostenol, treprostinil, iloprost), prostacyclin IP receptor (e.g., Selexipag), endothelin receptor antagonists (e.g., bosentan, ambrisentan, macitentan), PDE 5 inhibitors (e.g., sildenafil, tadalafil), soluble guanylate cyclase stimulator (e.g., riociguat), therapeutics for mitochondria dysfunction (e.g., bardoxolone methyl), anti-inflammation agents (e.g., rituximab, tocilizumab, ubenimex), and agents that modulate oxidative stress (e.g., dimethyl fumarate, intravenous iron).

In some embodiments, the one or more additional therapeutic agents include those useful, e.g., as therapeutics for heart failure or hypertension. Non-limiting examples include α-blockers (e.g., doxazosin, prazosin, tamsulosin, terazosin), β-blockers (e.g., acebutolol, acetutolol, atenolol, bisoprol, bupranolol, carteolol, carvedilol, celiprolol, esmolol, mepindolol, metoprolol, nadolol, oxprenolol, penbutolol, pindolol, propranolol, taliprolol), calcium channel blockers including but not limited to dihydropyridines (DHPs) (e.g., amlodipine, felodipine, isradipine, lacidipine, nicardipine, nifedipine, nigulpidine, nilutipine, nimodiphine, nisoldipine, nitrendipine, nivaldipine, ryosidine) and non-DHPs (e.g., anipamil, diltiazem, fendiline, flunarizine, gallpamil, mibefradil, prenylamine, tiapamil, verapamil), diurectics (e.g., thiazide derivatives such as, but not limited to, amiloride, chlorothalidon, chlorothiazide, hydrochlorthiazide, and methylchlorothiazide), centrally acting hypertensive agents (e.g., clonidine, guanabenz, guanfacine, methyldopa), angiotensin converting enzyme (ACE) inhibitors (alaceptril, benazepril, benazaprilat, captopril, ceronapril, cilazapril, delapril, enalapril, analaprilat, fosinopril, Lisinopril, moexipiril, moveltopril, perindopril, quinapril, quinaprilat, ramipril, ramiprilat, spriapril, temocapril, trendolapril, and zofenopril) and dual ACE/NEP inhibitors (e.g., omapatrilat, fasidotril, and fasidotrilat), angiotensin receptor blockers (ARBs) (e.g., candesartan, eprosartan, irbesartan, losartan, olmesartan, tasosartan, telmisartan, valsartan) and dual ARB/NEP inhibitors (e.g., combinations of valsartan and sacubitril), neutral endopeptidase (NEP) inhibitor (e.g., sacubitril), aldosterone synthase inhibitors (e.g., anastrozole, fadrozole, exemestane), endothelin antagonists (e.g., bosentan, enrasentan, atrasentan, darusentan, macitentan, sitaxentan, tezosentan), inhibitors of funny current (e.g., ivabradine), myosin activators (e.g., cardiac myosin activators), natriuretic, saluretic, vasodilator/vasorelaxation agents (e.g., nitrates), mineralocorticoid receptor antagonists, renin inhibitors, digitalis compounds, inotropic agents and β-receptor agonists, anti-hyperlipidemic agents, plasma HDL-raising agents, anti-hypercholesterolemic agents, cholesterol biosynthesis inhibitors (e.g., HMG CoA reductase inhibitors), LXR agonist, probucol, raloxifene, nicotinic acid, niacinamide, cholesterol absorption inhibitors, bile acid sequestrants (e.g., anion exchange resins, or quaternary amines such as cholestyramine or colestipol), low density lipoprotein receptor inducers, clofibrate, fenofibrate, bezafibrate, ciprofibrate, gemfibrizol, vitamins (e.g., vitamin B₆, vitamin B₁₂, anti-oxidant vitamins), platelet aggregation inhibitors, fibrinogen receptor antagonists, aspirin, and fibric acid derivatives.

In some embodiments, the one or more additional therapeutic agents include those useful, e.g., for treating diabetes. Non-limiting examples include sulfonylureas (e.g., chlorpropamide, tolbutamide, acetohexamide, tolazamide, glyburide, gliclazide, glynase, glimepiride, glipizide), biguanides (e.g., metformin), thiazolidinediones (e.g., ciglitazone, pioglitazone, troglitazone, rosiglitazone), insulin sensitizers related to the above (e.g., selective and non-selective activators of PPAR-alpha, PPAR-beta and PPAR-gamma), dehydroepiandrosterone (also referred to as DHEA or its conjugated sulfate ester, DHEA-SO₄), anti-glucocorticoids, TNF-alpha inhibitors, dipeptidyl peptidase IV (DPP4) inhibitors (e.g., sitagliptin, saxagliptin), GLP-1 agonists or analogs (such as exenatide), alpha-glucosidase inhibitors (such as acarbose, miglitol, and voglibose), pramlintide (a synthetic analog of the human hormone amylin), other insulin secretagogues (such as repaglinide, gliquidone, and nateglinide); and insulin.

In some embodiments, the one or more additional therapeutic agents include those useful, e.g., for treating obesity. Non-limiting examples include phenylpropanolamine, phentermine, diethylpropion, mazindol, fenfluramine, dexfenfluramine, phentiramine, beta3-adrenergic receptor agonist agents, sibutramine, gastrointestinal lipase inhibitors (e.g., orlistat), leptins, neuropeptide Y, enterostatin, cholecytokinin, bombesin, amylin, histamine H₃ receptors, dopamine D₂ receptor modulators, melanocyte stimulating hormone, corticotrophin releasing factor, galanin, and gamma amino butyric acid (GABA).

Other additional therapeutic agents include: but are not limited to, anti-atherosclerotic agents, anti-dyslipidemic agents, antihyperinsulinemic agents, anti-thrombotic agents, anti-retinopathic agents, anti-neuropathic agents, anti-nephropathic agents, anti-ischemic agents, anti-hyperlipidemic agents, anti-hypertriglyceridemic agents, anti-hypercholesterolemic agents, anti-restenotic-agents, anti-pancreatic agents, anorectic agents, memory enhancing agents, antidementia agents, cognition promoting agents, appetite suppressants, agents for treating peripheral arterial disease, agents for treating malignant tumors, anti-inflammatory agents, aquaretics, digoxin, nitric oxide donors, hydralazines, ionotropes, vasopressin receptor antagonists, statins, anti-arrhythmics, phosphodiesterase inhibitors (e.g., PDE5 inhibitors), and nephro-protectives.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

### EXAMPLES

### Example 1. Synthesis of Compound I

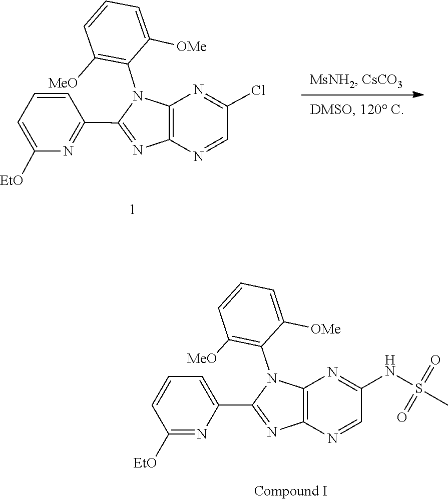 [SMILES: CCOc1cccc(-c2nc3ncc(Cl)nc3n2-c2c(OC)cccc2OC)n1 ; CCOc1cccc(-c2nc3ncc(NS(C)(=O)=O)nc3n2-c2c(OC)cccc2OC)n1]

To a solution of 1 (3.4 kg, 8.27 mol, 1 eq) in DMSO (18.49 kg) was added MsNH₂ (1.19 kg, 12.51 mol, 1.50 eq) and Cs2CO3 (8.06 kg, 24.74 mol, 3 eq) in four portions. The mixture was stirred at 120° C. for 20 hr. LCMS showed that 1 was consumed completely. The mixture was cooled to 20-30° C. and then diluted with water (120 L). The mixture was filtered and the filtration was acidified with aqueous HCl (3M) to pH of 3. The suspension was filtered and the filter cake was dissolved in THF (42.77 kg). After filtration, the solution was concentrated in reduced pressure to give crude product. The crude product was purified by trituration from ACN (38.0 kg) at 90° C. for 16 h to give Compound 1(1.93 kg), which was combined with 18 batches (obtained from totally 9.86 kg of 1), and re-crystallized from THF:ACN=1:5 (30 L) to afford Compound 1(5.1 kg, 10.85 mol, 45% yield). The material was confirmed by HPLC (Waters XBridge C18 4.6×150 mm, 3.5 μm; oven temp: °40 C; Flow rate: 1.0 mL/min; wavelength: 210 nm; Mobile phase A: 0.1% H₃PO₄ in H₂O, V/V; Mobile phase B: ACN) and ¹H NMR. Pd residue is 4 ppm. ¹H NMR (400 MHz, DMSO-d6): δ 11.05 (s, 1H), 8.31 (d, J=2.4 Hz, 1H), 7.96 (d, J=7.2 Hz, 1H), 7.86 (t, J=8.0 Hz, 1H), 7.46 (t, J=8.4 Hz, 1H), 6.85 (dd, J=8.4, 13.6 Hz, 3H), 3.58 (s, 6H), 3.40 (dd, J=7.2, 14.0 Hz, 2H), 3.21 (s, 3H), 1.19 (t, J=7.2 Hz, 3H).

Further analysis showed that the Compound I prepared by Example 1 was identified as Compound I free acid Form A.

### Example 2: Synthesis of Compound I Choline Salt

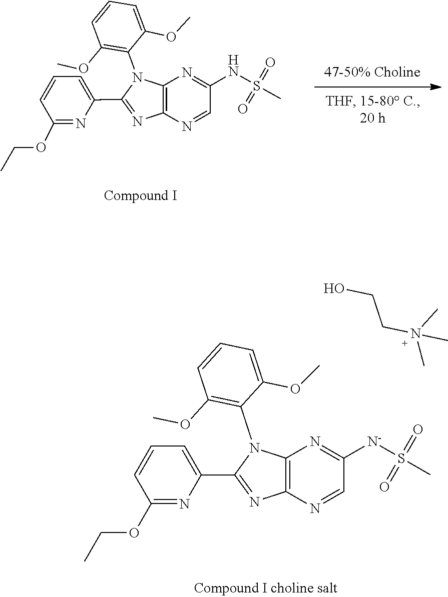

To a solution of Compound I (2.03 kg, 4.31 mol, 1.00 eq) in THF (24.00 kg), the mixture was heated to reflux, then 47-50% choline (1.28 kg, 5.17 mol, 49% purity, 1.20 eq) was dropwise at reflux and stirred for 4 h. The mixture was cooled to 15-20° C. and stirred for 16 h. The suspension was filtered and the filter cake (combined with 5 separate batches) was triturated with EtOH (14.00 L) at 80° C. for 16 h. Then cooled to 15-20° C. and the suspension was filtered and the filter cake was added in a vacuum drying oven (45° C., −0.092 Mpa, 16 h) and then dried to afford Compound I choline salt.

¹H NMR (400 MHz, DMSO-d6): δ 7.72-7.81 (m, 3H), 7.38 (t, J=8.4 Hz, 1H), 6.80 (t, J=4.4 Hz, 2H), 6.66 (dd, J=0.8, 8.0 Hz, 1H), 5.35 (s, 1H), 3.81-3.86 (m, 2H), 3.56 (s, 6H), 3.33-3.42 (m, 4H), 3.11 (s, 9H), 2.71 (s, 3H), 1.01 (t, J=7.2 Hz, 3H). LC-MS: positive scan found free form [M(C₂₁H₂₁N₆O₅S)+2H]⁺ 471.1 and choline (C₅H₄NO)⁺ 104.1; negative scan found free form M(C₂₁H₂₁N₆O₅S)⁻ 469.1.

### Example 3: Salt Screen

The starting material for the salt screen below was characterized by XRPD, TGA, and DSC. The XRPD pattern showed the material was crystalline, which was named as free acid Form A. The TGA/DSC results showed a weight loss of 0.6% up to 150° C. and an endotherm at 230.3° C. (peak temperature). Due to the small TGA weight loss, free acid Form A was postulated to be an anhydrate.

Approximate solubility of starting material (Compound I free acid Form A) was determined in 14 solvents at RT. Approximately 2 mg of sample was added into a 3-mL glass vial. Solvents in Table 1-3 were then added stepwise into the vials until the solids were dissolved visually or a total volume of 2 mL was reached. Solubility results summarized in Table 1-1 were used to guide the solvent selection in screening experiment design.

TABLE 1-1
Approximate solubility of starting material
(Compound I free acid Form A)

| Solvent | Solubility (mg/mL) | Solvent | Solubility (mg/mL) |
|---|---|---|---|
| MeOH | 1.0 < S < 2.1 | 2-MeTHF | S < 1.0 |
| EtOH | S < 1.1 | 1,4-dioxane | 2.0 < S < 5.0 |
| IPA | S < 0.9 | ACN | 2.0 < S < 5.0 |
| acetone | 2.1 < S < 5.2 | DCM | 5.3 < S < 10.5 |
| EtOAc | S < 1.0 | n-heptane | S < 1.3 |
| MTBE | S < 0.9 | toluene | S < 0.8 |
| THF | 9.0 < S < 18.0 | H₂O | S < 1.2 |

Using Compound I free acid Form A as starting material, a total of 32 salt screening experiments were performed using 8 bases in 4 solvent systems. Around 15 mg of starting material and equimolar corresponding salt formers were added in 0.5 mL solvent followed by slurry at RT for 4 days (the clear samples were transferred to slurry at −20° C. followed by evaporation at RT if still clear). The resulting suspension was centrifuged to retrieve the solids for XRPD test and the results were summarized in Table 1-2. A total of 7 crystalline salt hits and two new free acid polymorphs were obtained from salt screening and following experiments, which were characterized by XRPD, TGA, DSC and NMR or HPLC/IC.

TABLE 1-2
Summary of Salt Screening for Compound I

| | | A | B | C | DACN/H₂O |
|---|---|---|---|---|---|
| No. | Base | MeOH | Acetone | 1,4-dioxane | (9:1, v/v) |
| 1 | NaOH | Free acid Form A* | Na salt Form A | Na salt Form A | Free acid Form B* |
| 2 | KOH | K salt Form B* | K salt Form A | K salt Form A | K salt Form A** |
| 3 | Ca(OH)₂ | Base | Free acid Form | Gel** | Ca salt Form |
| | | | A + Base + peaks | | A + Base |
| 4 | Mg(OH)₂ | Free acid Form | Free acid Form | Base** | Mg salt Form |
| | | A + Base* | A + Base | | A + Base |
| 5 | Choline | Free acid Form A* | Choline salt | Choline salt Form B | Gel |
| | | | Form A | | |
| 6 | N-Methylglucamine | Gel** | Free acid Form A | Base | Gel** |
| 7 | Tris | Free acid Form A | Free acid Form | Base | Free acid Form B |
| | | | A + Base | | |
| 8 | Triethanolamine | Free acid Form A | Free acid Form A | Free acid Form A** | Free acid Form B |
| 9 | Blank | Free acid Form A | Free acid Form A | Free acid Form A** | Free acid Form B |

*This sample was obtained via slurry in −20° C.
**This sample was obtained via evaporation at RT.

### Instruments and Methods

### XRPD

For XRPD analysis, PANalytical Empyrean and X' Pert3 X-ray powder diffract meters were used. The XRPD parameters used are listed in Table 1-3.

TABLE 1-3
Parameters for XRPD test

| Parameters | Empyrean | X′ Pert3 | X′ Pert3 |
|---|---|---|---|
| X-Ray wavelength | Cu, Kα; | Cu, Kα; | Cu, Kα, |
| | Kα1 (Å): 1.540598 | Kα1 (Å): 1.540598 | Kα1 (Å):1.540598, |
| | Kα2 (Å): 1.544426 | Kα2 (Å): 1.544426 | Kα2 (Å):1.544426 |
| | intensity ratio | intensity ratio | Kα2/Kα1 intensity |
| | Kα2/Kα1: 0.50 | Kα2/Kα1: 0.50 | ratio: 0.50 |
| X-Ray tube setting | 45 kV, 40 mA | 45 kV, 40 mA | 45 kV, 40 mA |
| Divergence slit | Automatic | 1/8° | 1/8° |
| Scan mode | Continuous | Continuous | Continuous |
| Scan range (2θ/°) | 3~40 | 3~40 | 3~40 |
| Step size (2θ/°) | 0.0167 | 0.0263 | 0.0263 |
| Scan step time (s) | 17.780 | 46.665 | 46.665 |
| Test time | About 5 mins 30 s | About 5 mins | About 5 mins |

### TGA and DSC

TGA data were collected using a TA Q5000/Discovery 5500 TGA from TA Instruments. DSC was performed using a TA Q2000/Discovery 2500 DSC from TA Instruments. Detailed parameters used are listed in Table 1-4.

TABLE 1-4
Parameters for TGA and DSC test

| Parameters | TGA | DSC |
|---|---|---|
| Method | Ramp | Ramp |
| Sample pan | Aluminum, open | Aluminum, crimped/open |
| Temperature | RT- Target temperature | 25° C.- Target temperature |
| Heating rate | 10° C./min | 10° C./min |
| Purge gas | N₂ | N₂ |

### DVS

DVS was measured via a SMS (Surface Measurement Systems) DVS Intrinsic. The relative humidity at 25° C. were calibrated against deliquescence point of LiCl, Mg(NO₃)₂ and KCl. Parameters for DVS test are listed in Table 1-5.

### PLM

TABLE 1-5
Parameters for DVS test

| Parameters | Values | |
|---|---|---|
| Temperature | 25° | C. |
| Sample size | 10-20 | mg |
| Gas and flow rate | N2, 200 mL/min | |
| dm/dt | 0.002%/min | |
| Min. dm/dt stability duration | 10 | min |
| Max. equilibrium time | 180 | min |

| | |
|---|---|
| RH range | 0% RH-95% RH |
| RH step size | 10% (90% RH-0% RH-90% RH) |
| | 5% (95% RH-90% RH |
| | and 90% RH-95% RH) |

PLM picture was captured on Axio Lab. A1 upright microscope, purchased from Carl Zeiss German.

### Solution NMR

Solution NMR was collected on Bruker 400M NMR Spectrometer using DMSO-d6.

### HPLC/IC

Waters H-Class UPLC were utilized and detailed chromatographic conditions are listed in Table 1-6. IC parameters were listed in Table 1-7.

TABLE 1-6
HPLC methods for purity/solubility test

| Parameters | Waters H-Class with PDA detector | | | |
|---|---|---|---|---|
| Column | ACQUITY UPLC BEH C18 2.1 mm/50 mm/1.7 μm | | | |
| Mobile phase | | A: 0.2% FA in H₂0 | | |
| | | B: 0.2% FA in ACN | | |
| | Purity | | Solubility | |
| | Time (min) | % B | Time (min) | % B |
| Gradient table | 0.0 | 10 | 0.0 | 10 |
| | 0.5 | 10 | 0.5 | 10 |
| | 9.0 | 95 | 4.0 | 95 |
| | 11.5 | 95 | 6.0 | 95 |
| | 13.0 | 10 | 6.1 | 10 |
| | 15.0 | 10 | 8.0 | 10 |
| Run time | 15.0 min | | 8.0 min | |

| | |
|---|---|
| Flow rate | 0.3 mL/min |
| Injection volume | 1 μL |
| Detector wavelength | UV at 254, 280 and 214 nm |
| Column temperature | 40° C. |
| Sampler temperature | RT |
| Diluent | ACN |

TABLE 1-7
IC parameters for stoichiometric test

| IC | ThermoFisher ICS-1100 | |
|---|---|---|
| Column | IonPac AS18 Analytical Column (4 × 250 mm) | |
| Mobile phase | 25 mM methanesulfonic acid | |
| Injection volume | 25 | μL |
| Flow rate | 1.0 | mL/min |
| Cell temperature | 35° | C. |
| Column temperature | 35° | C. |
| Current | 80 | mA |

| | |
|---|---|
| Run time | 7.0 min for Na⁺⁻, 7.0 min for K⁺ |

### Characterization of Salt Forms

Seven salt forms were obtained from salt screening and further experiments, which were characterized by XRPD, TGA and DSC. The salt stoichiometry was determined using HPLC/IC or ¹H NMR. All the characterization results were summarized in Table 1-8.

TABLE 1-8
Characterization Summary of Compound I Salts

| | Weight loss | Endotherm | Molar ratio |
|---|---|---|---|
| Salt | (%, 150° C.) | (° C., peak) | (base/FA) |
| Na salt Form A | 3.6 | 92.9, 276.7 | 1.0 |
| K salt Form A | 3.7 | 74.2, 304.6 | 1.0 |
| K salt Form B | 8.4 | 303.6 | 1.0 |
| Ca salt Form A | 2.1*, 6.8^{#} | 114.2, 212.3 | 0.5 |
| Mg salt Form A | 12.0* | 136.6 | 0.5 |
| Choline salt Form A | 3.3 | 196.6 | 0.9 |
| Choline salt Form B | 5.9 | 74.4, 172.1 | 1.2 |
| Free acid Form B | 8.5 | 108.8, 230.4 | — |
| Free acid Form C | 3.1 | 229.9 | — |

*up to 120° C.;
^{#}from 120° C. to 200° C.

### Na Salt

Compound I sodium salt Form A (Na salt Form A) was obtained via slurry Compound I free acid Form A and equimolar NaOH in acetone at RT for 4 days. The XRPD pattern is displayed in FIG. 6A. The TGA/DSC curves of Compound I sodium salt Form A are displayed in FIG. 61B, which showed a weight loss of 3.6% up to 150° C. and two endotherms at 92.9 and 276.7° C. (peak). The ¹H NMR showed the molar ratio of acetone/API was 0.01 (0.1 wt %). HPLC/IC showed the molar ratio was 1.0 (base/FA).

### K Salt

Compound I potassium salt Form A (K salt Form A) was obtained via slurry free acid Form A and equimolar KOH in acetone at RT for 4 days. Compound I potassium salt Form B (K salt Form B) was obtained via a slurry of Compound I free acid Form A and equimolar KOH in MeOH at −20° C. for 4 days. The XRPD patterns are displayed in FIG. 7A and FIG. 8A, respectively.

The TGA/DSC curves of Compound I potassium salt Form A (K salt Form A) are displayed in FIG. 7B, which showed a weight loss of 3.7% up to 150° C. and two endotherms at 74.2° C. and 304.6° C. (peak). The H NMR showed the molar ratio of acetone/API was 0.03 (0.3 wt %). HPLC/IC results showed the molar ratio was 1.0 (base/FA).

The TGA/DSC curves of Compound I potassium salt Form B (K salt Form B) are displayed in FIG. 8B, which showed a weight loss of 8.4% up to 150° C. and an endotherm at 303.6° C. (peak). The ¹H NMR showed no residual MeOH was detected. HPLC/IC showed the molar ratio was 1.0 (base/FA).

### Ca Salt

Compound I calcium salt Form A (Ca salt Form A) was obtained via slurry free acid Form A and equimolar Ca(OH)₂ in ACN:H₂O (9:1, v/v) at RT for 4 days. The XRPD pattern showed residual Ca(OH)₂ was detected in this sample.

Another Compound I calcium salt Form A (Ca salt Form A) was obtained via slurry free acid Form A and Ca(OH)₂ (molar ratio of 0.5, base/acid) in ACN:H₂O (9:1, v/v) at RT for 1 day. The XRPD pattern is shown in FIG. 9A. The TGA/DSC curves of Compound I calcium salt Form A (Ca salt Form A) is shown in FIG. 9B, which showed a two-step weight loss of 2.1% up to 120° C. and 6.8% from 120° C. to 200° C., and two endotherms at 114.2 and 212.3° C. The ¹H NMR showed the molar ratio of ACN/API was 0.07 (0.6 wt %). HPLC/IC showed the molar ratio was 0.5 (base/FA).

### Mg Salt

Compound I magnesium salt Form A (Mg salt Form A) was obtained via slurry free acid Form A and equimolar Mg(OH)₂ in ACN:H₂O (9:1, v/v) at RT for 4 days. The XRPD pattern showed residual Mg(OH)₂ was detected.

Compound I magnesium salt Form A (Mg salt Form A) was obtained via slurry free acid Form A and Mg(OH)₂ (molar ratio of 0.5, base/acid) in ACN:H₂O (9:1, v/v) at RT for 2 days. The XRPD pattern is shown in FIG. 10A. The TGA/DSC curves of Compound I magnesium salt Form A (Mg salt Form A) is shown in FIG. 10B which showed a weight loss of 12.0% up to 120° C. and an endotherm at 136.6° C. (peak). The ¹H NMR showed the molar ratio of ACN/API was 0.71 (5.7 wt %). HPLC/IC showed the molar ratio was 0.5 (base/FA).

### Choline Salt

Compound I choline salt Forms A and B were obtained via slurry free acid Form A and equimolar choline in acetone and 1,4-dioxane, respectively, at RT for 4 days.

The TGA/DSC curves of choline salt Form A showed a weight loss of 3.3% up to 150° C. and an endotherm at 196.6° C. (peak). The ¹H NMR showed the molar ratio of choline/Compound I free acid was 0.9 and the molar ratio of acetone/API was 0.01 (0.1 wt %).

The TGA/DSC curves of choline salt Form B showed a weight loss of 5.9% up to 150° C. and two endotherms at 74.4° C. and 172.1° C. (peak). The ¹H NMR showed the molar ratio of choline/Compound I free acid was 1.2 and the molar ratio of 1,4-dioxane/API was 0.7 (6.2 wt %).

### Free Acid

Compound I free acid Form B (Free acid Form B) was obtained via slurry free acid Form A in ACN:H₂O (9:1, v/v) at RT for 4 days. The XRPD is shown in FIG. 4A.

The TGA/DSC curves of Compound I free acid Form B (free acid Form B) is shown in FIG. 4B, which showed a weight loss of 8.5% up to 150° C. and two endotherms at 108.8° C. and 230.4° C. (peak). The ¹H NMR showed the molar ratio of ACN/API was 0.8 (6.1 wt %).

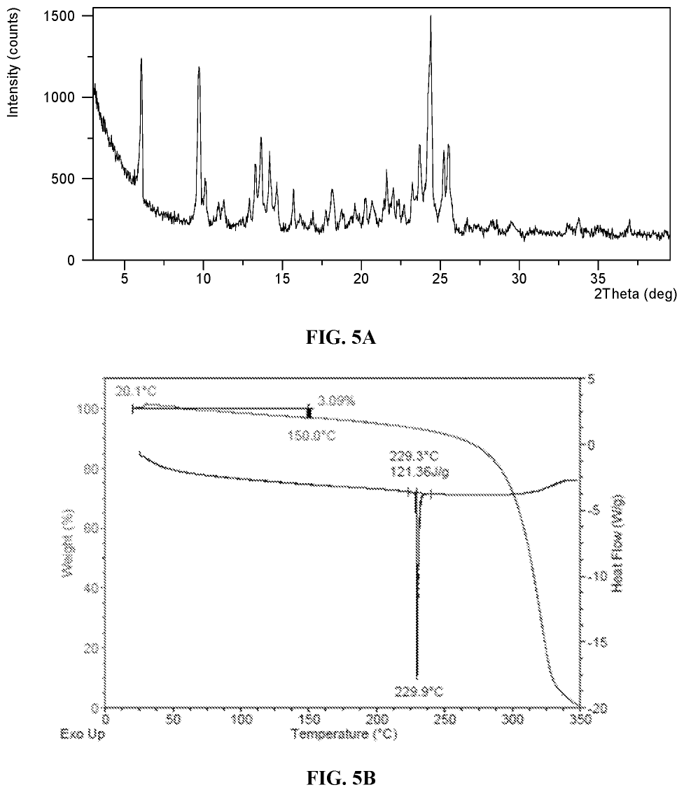

Compound I free acid Form C (Free acid Form C) was obtained via heating free acid Form B to 150° C. and cooling to RT. The XRPD patterns is shown in FIG. 5A.

The TGA/DSC curves of Compound I free acid Form C (Free acid Form C) were displayed in FIG. 5B, which showed a weight loss of 3.1% up to 150° C. and an endotherm at 229.9° C. (peak). The ¹H NMR showed no residual ACN was detected.

### Re-Preparation of Selected Salts

Considering the small TGA weight loss and sharp DSC endotherms, Na salt Form A, K salt Form A and choline salt Form A were selected for re-preparation at 300 mg scale. All the samples were successfully re-prepared and characterized by XRPD, TGA, DSC, NMR or HPLC/IC, which were summarized in Table 1-9.

TABLE 1-9
Summary of characterization for re-prepared salts

| | Weight loss | Endotherm | Molar ratio |
|---|---|---|---|
| Sample form | (%, 150° C.) | (° C., peak) | (base/FA) |
| Na salt Form A | 3.8 | 89.2, 278.1 | 1.0 |
| K salt Form A | 2.3 | 74.2, 306.1 | 0.9 |
| Choline salt Form A | 1.9 | 196.6 | 1.0 |

### Na Salt

Compound I sodium salt Form A (Na salt Form A) was re-prepared via slurry ˜300 mg Compound I free acid Form A and equimolar NaOH in acetone at RT for 24 hrs. The XRPD pattern is displayed in FIG. 6A. The TGA/DSC curves of Na salt Form A is displayed in FIG. 6B which showed a weight loss of 3.8% up to 150° C. and two endotherms at 89.2 and 278.1° C. (peak). HPLC/IC results showed the molar ratio was 1.0 (base/FA). Due to the step TGA weight loss and corresponding DSC endotherm, Na salt Form A was postulated to be a hydrate.

### K Salt

Compound I potassium salt Form A (K salt Form A) was re-prepared via slurry ˜300 mg Compound I free acid Form A and equimolar KOH in acetone at RT for 48 hrs. The XRPD pattern was displayed in FIG. 7A. The TGA/DSC curves of K salt Form A were displayed in FIG. 7B, which showed a weight loss of 2.3% up to 150° C. and two endotherms at 74.2 and 306.1° C. (peak). HPLC/IC results showed the molar ratio was 0.9 (base/FA). Due to the step TGA weight loss and corresponding DSC endotherm, K salt Form A was postulated to be a hydrate.

### Choline Salt

Compound I choline salt Form A (choline salt Form A) was re-prepared via slurry ˜300 mg free acid Form A and equimolar choline in acetone at RT for 24 hrs. The XRPD pattern is shown in FIG. 1A. The TGA/DSC curves of choline salt Form A is displayed in FIG. 1B, which showed a weight loss of 1.9% up to 150° C. and an endotherm at 196.6° C. (peak). The ¹H NMR showed the molar ratio of base/FA was 1.0 and the molar ratio of acetone/API was 0.01 (0.1 wt %). Due to the small TGA weight loss, Compound I choline salt Form A was postulated to be an anhydrate.

### Salt Evaluation

Three re-prepared salt samples were used for salt evaluation, including hygroscopicity, kinetic solubility and solid stability. The starting material Compound I free acid Form A was also evaluated for comparison.

### Hygroscopicity

In order to evaluate the hygroscopicity of Compound I free acid Form A and re-prepared salt forms, DVS isotherm plots were collected at 25° C. between 0% RH and 95% RH. XRPD characterization was performed for the samples after DVS test. The DVS evaluation results are summarized in Table 1-10. Based on the results, the lowest water uptake was observed for Compound I free acid Form A. Compound I Na salt Form A converted to a new form after DVS test. No significant difference was observed between Compound I K salt Form A and Compound I choline salt Form A.

TABLE 1-10
Summary of DVS evaluation

| | Water uptake | Form change |
|---|---|---|
| Form | (25° C./80% RH) | after DVS |
| Na salt Form A | 1.13% | Yes* |
| K salt Form A | 3.71% | No |
| Choline salt Form A | 3.85% | No |
| Free acid Form A | 0.03% | No |

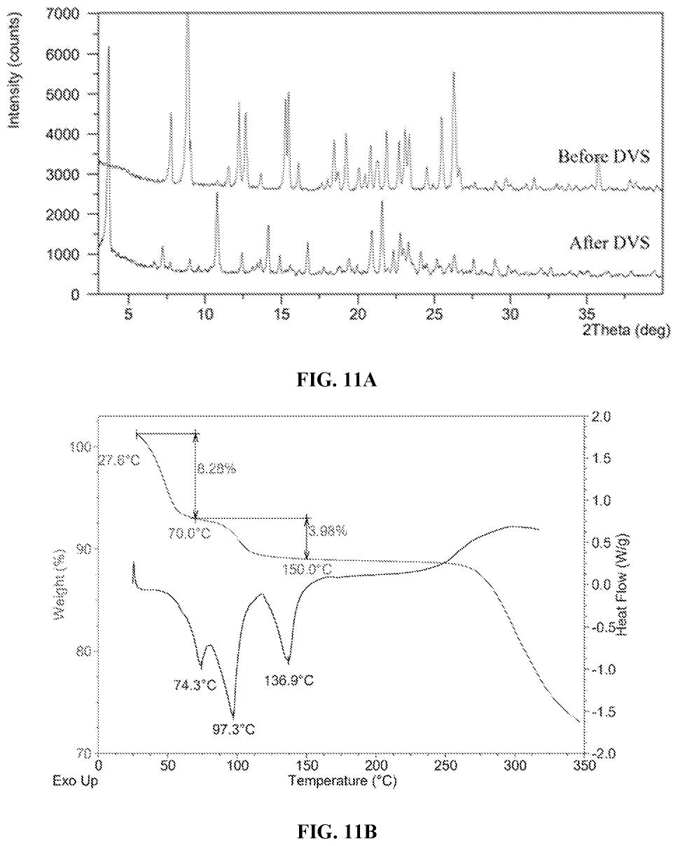

*Na salt Form A converted to a new form, which was named as potential Compound I sodium salt Form B (FIG. 11A). The TGA/DSC curves showed a weight loss of 12.3% up to 150° C. and three endotherms at 74.3, 97.3 and 196.6° C. (peak) (FIG. 11B).

### Kinetic Solubility

Kinetic solubility was measured for Compound I free acid Form A and re-prepared salt forms in water and three bio-relevant media.

Around 20 mg material was weighed into 4 mL water, SGF, FaSSIF or FeSSIF¹ followed by rolling at 25 rpm at 37° C. for 1, 2, 4 and 24 hrs. At each time point, around 0.8 mL suspension was sampled out for centrifugation and filtration. Solids were tested by XRPD and filtrates were tested by HPLC and pH. Based on the results, all salts exhibited similar solubility profiles and the solubility of salts was higher than free acid Form A in water and FaSSIF, which was postulated to be due to pH difference. XRPD results showed disproportionation was observed for all salt samples after solubility study.

### Solid Stability

Compound I Free acid Form A and re-prepared salt forms were placed under the conditions of 25° C./60% RH and 40° C./75% RH for one week for solid stability evaluation. The physical and chemical stability were evaluated by XRPD and HPLC purity, respectively. Based on the results, no form change or obvious HPLC purity decrease was observed for all samples.

### Conclusion

Using Compound I free acid Form A as starting material, a salt screening was performed under 32 conditions using 8 bases in 4 solvent systems. A total of 7 crystalline salt hits and 2 new free acid forms were obtained. Based on the characterization results (small TGA weight loss and sharp endothermic signal on DSC curve), Compound I sodium salt Form A, Compound I potassium salt Form A, and Compound I choline salt Form A were selected for re-preparation, which were all re-prepared successfully.

The re-prepared salts were used for salt evaluation and Compound I free acid Form A was also evaluated for comparison. The DVS results showed that free acid Form A exhibited the lowest water uptake and form change was observed after DVS for Compound I Na salt Form A. The kinetic solubility results showed that all salts exhibited similar solubility profiles and the solubility of salts was higher than free acid in H₂O and FaSSIF. The results of solid stability evaluation showed no form change or obvious HPLC purity decrease was observed for all samples after stored at 25° C./60% RH or 40° C./75% RH for one week. Based on the salt evaluation and solid-state characterization results, Compound I choline salt Form A was selected to be re-prepared at 2 g scale for further PK study.

Combining the results of salt evaluation and PK study, Compound I choline salt was selected for further polymorph screening.

### Re-preparation of Choline Salt

Compound I choline salt Form A was re-prepared at 2 g scale and the procedures were listed below. The sample was characterized by XRPD, TGA, DSC, and NMR. The XRPD pattern is FIG. 1A. The TGA/DSC curves of Compound I choline salt Form A is FIG. 1B, which showed a weight loss of 2.9% up to 150° C. and an endotherm at 195.6° C. (peak). ¹H NMR showed the molar ratio of base/FA was 1.0 and the molar ratio of acetone/API was 0.03 (0.3 wt %).
1. Weigh ˜1.05 g choline (49 wt %, 4.25 mmol) into a 100-mL reactor;
2. Add 30 mL acetone into the reactor to obtain a suspension;
3. Weigh ˜2.00 g free acid (4.25 mmol) into the reactor;
4. Stir at RT for ˜24 hrs;
5. Filter the suspension and dry the solid at RT under vacuum (˜1.5 g product was obtained).

### Example 4: Polymorph Screening and Evaluation for Compound I Choline Salt

The purpose of this project was to perform polymorph screening and evaluation for choline salt to select a lead form for further study. The starting material was characterized by XRPD, TGA and DSC. XRPD pattern showed the material was free acid Form A. The TGA/DSC results showed a weight loss of 0.5% up to 150° C. and an endotherm at 231.1° C. (peak temperature). Due to the small TGA weight loss, free acid Form A was postulated to be an anhydrate.

### Instruments and Methods

The XRPD, TGA, DSC, PLM, NMR, and HPLC instruments and methods used were the same as in Example 3.

### Summary

14 slurry experiments were performed at 50° C. for re-prepared choline salt Form A and free acid Form A respectively. XRPD results showed that only choline salt Form A and free acid Form A were obtained from the polymorph screening experiments.

The re-prepared choline salt Form A was used for evaluation, including solubility and solid stability. Free acid Form A was selected as comparison for pH solubility and solid stability evaluation.

The pH solubility results showed that choline salt Form A exhibited higher solubility than free acid Form A in pH buffers.

The results of solid stability evaluation showed no form change or obvious HPLC purity decrease for choline salt Form A and free acid Form A after stored at 25° C./60% RH or 40° C./75% RH for 54 and 91 days.

The evaluation results showed choline salt Form A was physically/chemically stable under the conditions of 25° C./60% RH and 40° C./75% RH for 91 days and exhibited higher solubility than free acid Form A in pH buffers.

### Re-Preparation of Choline Salt Form A

Compound I choline salt Form A was re-prepared via slurry 3.5 g Compound I free acid Form A and equimolar choline in acetone at RT for 4 days. The XRPD pattern was shown in FIG. 1A. The TGA/DSC curves of choline salt Form A were displayed in FIG. 1B, which showed a weight loss of 1.9% up to 150° C. and an endotherm at 195.6° C. (peak). The ¹H NMR showed the molar ratio of base/FA was 1.0 and the molar ratio of acetone/API was 0.04 (0.4 wt %). PLM image showed choline salt Form A was irregular particles with size at about 10 μm. Compound I choline salt Form A was shown to have good solubility in polar solvents, including methanol, DMSO, and water, and was less soluble in certain non-polar solvents, such as IPA, acetone, MEK, MIBK, ethyl acetate, isopropyl acetate, MTBE, THF, 2-MeTHF, CHCl₃, n-heptane, toluene, DCM, and 1,4-dioxane.

### Polymorph Screening

14 slurry experiments were performed at 50° C. for choline salt Form A and free acid Form A, respectively, to preliminarily investigate the polymorph risk of choline salt and free acid. About 15 mg of choline salt Form A was suspended in 0.5 mL of the corresponding solvent in an HPLC vial. After the suspension was magnetically stirred (˜750 rpm) for about 3 days at 50° C., the obtained solid form was choline salt Form A and results are summarized in Table 4-1. About 15 mg of free acid Form A was suspended in 0.5 mL of the corresponding solvent in an HPLC vial. After the suspension was magnetically stirred (˜750 rpm) for about 2 days at 50° C., the obtained solid form was free acid Form A and results are summarized in Table 4-2.

TABLE 4-1
Summary of slurry experiments at 50° C. for choline salt Form A

| Solvent (v/v) | Results |
|---|---|
| IPA | Choline salt Form A |
| Acetone | Choline salt Form A |
| IPAc | Choline salt Form A |
| 1,4-dioxane | Choline salt Form A |
| MTBE | Choline salt Form A |
| 2-MeTHF | Choline salt Form A |
| Anisole | Choline salt Form A |
| Toluene | Choline salt Form A |
| 1,4-dioxane:ACN (1:1) | Choline salt Form A |
| CHCl₃ | Choline salt Form A |
| MIBK | Choline salt Form A |
| n-heptane | Choline salt Form A |
| MeOH/toluene (1:9) | Choline salt Form A |
| Acetone/H₂O (19:1) | Choline salt Form A |

### Solution Crystallization of Compound I Choline Salt Form A

TABLE 4-2
Summary of slurry experiments at 50° C. for free acid Form A

| Solvent (v/v) | Results |
|---|---|
| IPA | Free acid Form A |
| Acetone | Free acid Form A |
| IPAc | Free acid Form A |
| 1,4-dioxane | Free acid Form A* |
| MTBE | Free acid Form A |
| 2-MeTHF | Free acid Form A |
| Anisole | Free acid Form A |
| Toluene | Free acid Form A |
| ACN | Free acid Form A |
| CHCl₃ | Free acid Form A |
| MIBK | Free acid Form A |
| n-heptane | Free acid Form A |
| MeOH/toluene (1:9) | Free acid Form A |
| Acetone/H₂O (19:1) | Free acid Form A |

*Clear solution was obtained at 50° C. and suspension was obtained after transferred to slurry at RT.

Compound I choline salt Form A was re-prepared via crystallization at ˜100 mg scale in THF and CHCl₃ at RT. The procedure were listed in Table 4-3. XRPD showed Compound I choline salt Form A was successfully obtained via solution crystallization in THF and CHCl₃.

TABLE 4-3
Procedure of solution crystallization

| Sample form | Procedure |
|---|---|
| Choline salt | 1. Weigh ~99.3 mg free acid (0.21 mmol) into a 20-mL vial followed by addition of |
| Form A | 5 mL THF at RT to obtain a clear solution; |
| | 2. Add 62.1 mg choline solution (aq, 48%~50%, 0.25 mmol) into the vial; |
| | 3. Two layers were observed, which turned turbid in a few minutes; |
| | 4. Add a few choline salt Form A seed into the vial; |
| | 5. Stir at RT for ~18 hrs and yellow suspension was obtained; |
| | 6. Filter the suspension and dry the solid at RT. |
| Choline salt | 1. Weigh ~100.1 mg free acid (0.21 mmol) into a 20-mL vial followed by addition of |
| Form A | 9 mL CHCl₃ at 50° C. to obtain a clear solution; |
| | 2. Add 59.8 mg choline solution (aq, 48%~50%, 0.24 mmol) into the vial at 50° C.; |
| | 3. Clear solution was obtained and then transferred to stir at RT; |
| | 4. Add a few Compound I choline salt Form A seed into the vial (not dissolved); |
| | 5. Stir at RT for ~18 hrs and yellow suspension was obtained. |
| | 6. Filter the suspension and dry the solid at RT. |

### Conclusion

14 slurry experiments were performed at 50° C. for re-prepared Compound I choline salt Form A and free acid Form A respectively. XRPD results showed that only Compound I choline salt Form A and free acid Form A were obtained from the polymorph screening experiments.

The re-prepared Compound I choline salt Form A was used for evaluation, including solubility and solid stability evaluation. Free acid Form A was selected as comparison for pH solubility and solid stability evaluation.

The pH solubility results showed that Compound I choline salt Form A exhibited higher solubility than free acid Form A in pH buffers.

The results of solid stability evaluation showed no form change or obvious HPLC purity decrease for Compound I choline salt Form A and free acid Form A after stored at 25° C./60% RH or 40° C./75% RH for 54 and 91 days.

The evaluation results showed Compound I choline salt Form A was physically/chemically stable under the conditions of 25° C./60% RH and 40° C./75% RH for 91 days and exhibited higher solubility than free acid Form A in pH buffers.

Compound I choline salt Form A exhibited desired solid-state properties over other salts/solid forms tested. For example, sodium salt exhibited hygroscopicity and was less stable after exposed to high humidity when compared to Compound I choline salt Form A. Compound I magnesium salt Form A, as well as the Compound I calcium salts and potassium salts were solvates, whereas Compound I choline salt Form A is considered to be a stable non-stoichiometric hydrate with a reasonable melting point.

### Example 5: Polymorph Screen and Single Crystal Structure Determination for Compound I Choline Salt

This project includes a polymorph screening and a single crystal study for Compound I choline salt Form A. The purpose of the polymorph screening was to search for potential crystal forms and to determine if Compound I choline salt Form A was the suitable form for further pharmaceutical development. The purpose of the single crystal study was to understand the crystal form of Compound I choline salt Form A and to confirm whether it was a salt or co-crystal.

The starting material as received was characterized by X-ray powder diffraction (XRPD), polarized light microscopy (PLM), thermogravimetric analysis (TGA), differential scanning calorimetry (DSC), and proton nuclear magnetic resonance (¹H NMR). The characterization results indicated that the starting material was crystalline, defined as Compound I choline salt Form A, and was a mono-choline salt (free acid:choline=1:1).

Using Form A as starting material, a polymorph screening and single crystal cultivation was conducted under 103 conditions through methods of anti-solvent addition, solid vapor diffusion, liquid vapor diffusion, evaporation, slurry at RT/50° C., slow cooling, polymer induced crystallization and grinding. Based on the characterization results, only Compound I choline salt Form A was obtained with its potential single crystals observed via liquid vapor diffusion.

Single crystal X-ray diffraction (SCXRD) was conducted. The single crystal structure determination confirmed the chemical structure of Compound I choline salt, showing Form A was a non-stoichiometric hydrate of mono-choline salt.

Based on the polymorph screening and single crystal study results above, the only form Compound I choline salt Form A was recommended for further pharmaceutical development.

### Instruments and Methods

### X-ray Powder Diffraction (XRPD)

X-ray powder diffraction data were collected under ambient conditions on a Bruker D2 PHASER diffractometer with a low power X-ray generator of 300 W. Powder patterns were collected on a zero background sample holder with a 0.15 s/step with a total step of 1837, two theta at 0.02° per step at 30 kV and 10 mA. The X-ray tube of Cu (Kα) was employed, with the Kα2/Kα1 intensity ratio of 0.50 (1.54439 Å/1.5406 Å).

### Thermogravimetric Analysis (TGA)

Thermogravimetric analysis data were collected with a TA Discovery 550 series TGA. A few milligrams of material were heated from room temperature to target temperature with a heating rate of 10° C. per minute under nitrogen protection.

### Differential Scanning Calorimetry (DSC)

Differential scanning calorimetry was performed with a TA Discovery 2500 series DSC using approximately a few milligrams of material in a Tzero aluminum pan sealed with a Tzero hermetic lid. Samples were analyzed using a heating rate of 10° C. per minute under 50 mL per minute of nitrogen flow.

### Polarized Light Microscopy (PLM)

Photomicrographs were taken using an Olympus BX53M polarized light microscope at room temperature.

### Proton Nuclear Magnetic Resonance (HNMR)

¹H NMR data was taken using Agilent VNMR 400MR in D₂O solvent.

### Single Crystal X-ray Diffraction (SCXRD)

Single crystal X-ray diffraction data was collected on a Rigaku mm007 Saturn70 (Mo/Kα X-ray radiation, λ=0.71073 Å) diffractometer with Saturn70 detector, fixed-chi goniometer and Rigaku GN2 cryosystem.

### Solvent Abbreviation

The abbreviations of solvents used were listed in Table 5-1.

TABLE 5-1
Solvent abbreviation list

| Abbre- | | Abbre- | |
|---|---|---|---|
| viation | Solvent | viation | Solvent |
| H₂O | Water | THF | Tetrahydrofuran |
| MeOH | Methanol | 2-MeTHF | 2-Methyltetrahydrofuran |
| EtOH | Ethanol | ACN | Acetonitrile |
| IPA | Isopropanol | DCM | Dichloromethane |
| MEK | 2-Butanone | CHCl₃ | Trichloromethane |
| MIBK | Methyl isobutyl ketone | CH₂Br₂ | Dibromomethane |
| EtOAc | Ethyl acetate | DMAc | N,N-Dimethylacetamide |
| IPAc | Isopropyl acetate | DMSO | Dimethyl sulfoxide |
| MTBE | Methyl tert-butyl ether | DMF | N,N-Dimethylformamide |
| CPME | Cyclopentyl methyl ether | — | — |

### Starting Material Characterization

The starting material (Compound I choline salt Form A) was yellow in color with chemical purity of 99.9 area % and was analyzed by XRPD, PLM, TGA and DSC as received. The XRPD data showed it was Compound I choline salt Form A. Birefringent crystals were observed by PLM. TGA exhibited a weight loss of 1.3 wt % before 180.0° C. and DSC exhibited a sharp melting peak at 199.7° C. (peak) along with a broad endotherm at 104.0° C. (peak). ¹H NMR (D₂O) indicated a stoichiometry (free acid:choline) of 1:1 and no solvent signals observed.

### Approximate Solubility

The solubility of starting material (Compound I choline salt Form A) was estimated at room temperature (RT, ˜26° C.). Specifically, approximate 2 mg solid was added into HPLC glass vials. Solvents in Table 5-2 were then added stepwise (50/50/200/700 μL) into the vials until the solids were dissolved or a total volume of 1.0 mL was reached. The solubility data were used to guide the solvent selection in polymorph screening and single crystal cultivation.

TABLE 5-2
Approximate solubility of starting material at RT

| | Solubility | Solvent | Solubility |
|---|---|---|---|
| Solvent | (mg/mL) | (v:v) | (mg/mL) |
| H₂O | S > 43.0 | n-Heptane | S < 2.1 |
| MeOH | S > 45.2 | IPA | S < 2.2 |
| DMSO | 18.8 < S < 37.6 | Toluene | S < 2.2 |
| DMF | 20.2 < S < 40.4 | MIBK | S < 2.3 |
| EtOH | 6.8 < S < 20.5 | Acetone | S < 2.4 |
| ACN | 7.1 < S < 21.4 | Acetone/H₂O (19:1) | 7.2 < S < 21.6 |
| EtOAc | S < 1.7 | ACN/IPAc (1:1) | S < 2.1 |
| 2-Butanol | S < 1.8 | ACN/IPAc (9:1) | 2.2 < S < 7.3 |
| CPME | S < 1.8 | ACN/Toluene (1:1) | 2.1 < S < 7.0 |
| THF | S < 1.8 | ACN/Toluene (9:1) | 6.3 < S < 18.8 |
| 2-MeTHF | S < 1.8 | EtOH/H₂O (19:1) | 6.6 < S < 19.9 |
| MEK | S < 2.0 | IPA/ACN (1:1) | 7.0 < S < 21.0 |
| IPAc | S < 2.0 | MeOH/DCM (1:1) | S > 39.6 |
| MTBE | S < 2.1 | MeOH/DCM (1:19) | S > 38.6 |
| 1,4-Dioxane | S < 2.1 | MeOH/DCM (1:9) | S > 40.8 |
| DCM | S < 2.1 | THF/H₂O (19:1) | S > 40.4 |
| CHCl₃ | S < 2.1 | — | — |

### Polymorph Screening and Single Crystal Cultivation

A polymorph screening and single crystal cultivation were conducted under 103 conditions using starting material (Compound I choline salt Form A) through methods of anti-solvent addition, solid vapor diffusion, liquid vapor diffusion, slow evaporation, slurry at RT/50° C., slow cooling, polymer induced crystallization and grinding.

The crystallization methods and the results are summarized in Table 5-3. Based on the characterization results, Compound I choline salt Form A was obtained with its single crystals observed from four conditions via liquid vapor diffusion.

TABLE 5-3
Polymorph screening and single crystal cultivation summary of Compound I choline salt

| | | Experiment | |
|---|---|---|---|
| No. | Methods | No. | Result |
| 1 | Anti-solvent addition | 17 | Compound I choline salt Form A, gel |
| | | | (amorphous) |
| 2 | Solid vapor diffusion | 9 | Compound I choline salt Form A |
| 3 | Liquid vapor diffusion | 10 | Compound I choline salt Form A |
| | | | [single crystal], gel |
| 4 | Slow evaporation | 6 | Gel, oil |
| 5 | Slurry at RT | 22 | Compound I choline salt Form A |
| 6 | Slurry at 50° C. | 18 | Compound I choline salt Form A |
| 7 | Slow cooling | 12 | Compound I choline salt Form A, gel |
| 8 | Polymer-induced crystallization | 6 | Compound I choline salt Form A, gel |
| 9 | Grinding | 3 | Compound I choline salt Form A |
| Total | — | 103 | Compound I choline salt Form A, gel, |
| | | | oil |

### Anti-Solvent Addition

A total of 17 anti-solvent addition experiments were carried out at RT. Stock solutions with corresponding solvents below were prepared. Certain amount of starting material (Compound I choline salt Form A) were dissolved in the solvents to get solutions/suspensions which were filtered using a Nylon membrane (pore size of 0.22 μm). The filtrates were divided for each experiment with ˜50 mg starting material. The filtrates were magnetically stirred followed by addition of 0.2˜0.5 mL anti-solvent stepwise till precipitate appeared or the total amount of anti-solvent reached 15.0 mL. The obtained precipitates were isolated for XRPD analysis. Results in Table 5-4 showed that only one crystal form Compound I choline salt Form A was obtained.

TABLE 5-4
Summary of anti-solvent addition experiments

| Solvent | Anti-solvent | Result |
|---|---|---|
| MeOH | MIBK | Compound I choline salt Form A |
| | EtOAc | Compound I choline salt Form A |
| | MTBE | Compound I choline salt Form A |
| DMSO | EtOH | Compound I choline salt Form A + gel^{#} |
| | IPAc | Compound I choline salt Form A |
| | CPME | Compound I choline salt Form A |
| | Toluene | Compound I choline salt Form A |
| DMF | 2-Butanol | Compound I choline salt Form A* |
| | MIBK | Compound I choline salt Form A |
| | CHCl₃ | Gel^{#} |
| | MTBE | Compound I choline salt Form A |
| NMP | ACN | one peak + gel (amorphous)^{#} |
| | EtOAc | Compound I choline salt Form A |
| | MTBE | Compound I choline salt Form A |
| | DCM | Gel^{#} |
| ACN | CPME | Compound I choline salt Form A |
| | CHCl₃ | Gel |

^{#}Gels and/or trace of solids were obtained via vacuum drying at 60° C. after anti-solvent addition followed by slurry at RT/5° C./−5° C./−20° C. for 15 hrs/7 hrs/17 hrs/54.5 hrs and evaporation at RT for 10 days. Solids were characterized via XRPD indicating that the sample was Form A with low crystallinity and an amorphous sample was with one peak at 4.9° (20) which possibly from Form A.
*Solids were obtained via slurry at −5° C. after anti-solvent addition and slurry at RT/5° C. for 15 hrs/7 hrs.

### Solid Vapor Diffusion

Solid vapor diffusion experiments were conducted using nine different solvents. Approximate 25 mg of starting material (Compound I choline salt Form A) was weighed into 4-mL vials, which were placed into 20-mL vials with ˜3 mL of volatile solvent. The 20-mL vials were sealed with caps and kept at RT for 8˜10 days allowing solvent vapor to interact with sample. The solids were tested by XRPD and the results are shown in Table 5-5.

TABLE 5-5
Summary of solid vapor diffusion experiments

| Solvent | Result |
|---|---|
| IPA | Compound I choline salt Form A |
| EtOAc | Compound I choline salt Form A |
| MTBE | Compound I choline salt Form A |
| 2-MeTHF | Compound I choline salt Form A |
| ACN | Compound I choline salt Form A |
| Toluene | Compound I choline salt Form A |
| CHCl₃ | Compound I choline salt Form A |
| DMSO | Compound I choline salt Form A |
| H₂O | Compound I choline salt Form A |

### Liquid Vapor Diffusion

Liquid vapor diffusion experiments were conducted under 10 solvent conditions. 20˜50 mg of starting material (Compound I choline salt Form A) was dissolved in 0.3˜0.5 mL corresponding solvents. The solutions/suspensions were filtered using a Nylon membrane (pore size of 0.22 μm) and then the filtrates were collected in 4-mL vials which were covered by lids with one pinhole and were placed into 20-mL vials with ˜4 mL of volatile solvent. The 20-mL vials were sealed with caps and kept at RT for 1˜23 days allowing solvent vapor to interact with samples. The solids were tested by XRPD and the results in Table 5-6 showed that only Compound I choline salt Form A was obtained with 4 of them being potential single crystals as shown by PLM.

TABLE 5-6
Summary of liquid vapor diffusion experiments

| Solvent (v:v) | Anti-solvent | Result |
|---|---|---|
| MeOH | MTBE | Compound I choline salt Form A |
| EtOH | n-Hexane | Compound I choline salt Form A |
| | | [single crystal] |
| EtOH | DCM | Gel |
| DMSO | EtOAc | Compound I choline salt Form A |
| | | [single crystal] |
| DMSO | CHCl₃ | Gel |
| DMAc | MTBE | Compound I choline salt Form A |
| | | [single crystal] |
| DMF | DCM | Gel |
| NMP | MTBE | Compound I choline salt Form A |
| | | [single crystal] |
| ACN | DCM | Gel |
| THF/H₂O (19:1) | Acetone | Gel |

Gel: Gels were obtained via vacuum drying at 60° C. after liquid vapor diffusion for 14 days.

### Slow Evaporation

40˜50 mg of starting material (Compound I choline salt Form A) was dissolved in 2˜5 mL of solvents in 4-mL glass vials. Solutions/suspensions were filtered using a Nylon membrane (pore size of 0.22 in). The filtrates were covered by lids with 1 pinhole and subjected to evaporation at RT. Results summarized in Table 5-7 indicated that only gels/oil were observed.

TABLE 5-7
Summary of slow evaporation experiments

| Solvent (v:v) | Result |
|---|---|
| EtOH | Gel |
| ACN | Gel |
| MeOH/Toluene (1:1) | Gel |
| MeOH/Anisole (1:1) | Oil |
| MeOH/MIBK (1:1) | Gel |
| MeOH/IPAc (1:1) | Gel |

### Slurry at RT

Slurry experiments were conducted at RT in 22 solvents/mixtures. About 50 mg of starting material (Compound I choline salt Form A) was suspended in 0.5 mL of solvents/mixtures in HPLC vials. After the suspensions were stirred magnetically for 5 days at RT, the remaining solids were isolated for XRPD analysis. Results are summarized in Table 5-8.

TABLE 5-8
Summary of slurry experiments at RT

| Solvent (v:v) | Result |
|---|---|
| EtOH | Compound I choline salt Form A |
| IPA | Compound I choline salt Form A |
| Acetone | Compound I choline salt Form A |
| MEK | Compound I choline salt Form A |
| EtOAc | Compound I choline salt Form A |
| IPAc | Compound I choline salt Form A |
| MTBE | Compound I choline salt Form A |
| THF | Compound I choline salt Form A |
| 2-MeTHF | Compound I choline salt Form A |
| Anisole | Compound I choline salt Form A |
| 1,4-Dioxane/n-Hexane (3:1) | Compound I choline salt Form A |
| IPA/ACN (1:1) | Compound I choline salt Form A |
| ACN/CPME (1:1) | Compound I choline salt Form A |
| DCM/n-Heptane (3:1) | Compound I choline salt Form A |
| DMF/Toluene (1:5) | Compound I choline salt Form A |
| IPA/H₂O (a_{w} = 0.2, 982:18) | Compound I choline salt Form A |
| IPA/H₂O (a_{w} = 0.4, 956:44) | Compound I choline salt Form A |
| IPA/H₂O (a_{w} = 0.6, 919:81) | Compound I choline salt Form A |
| IPA/H₂O (a_{w} = 0.8, 848:152) | Compound I choline salt Form A |
| Acetone/H₂O (19:1) | Compound I choline salt Form A |
| H₂O | Syrup-like solution |
| 1,4-Dioxane | Compound I choline salt Form A |

### Slurry at 50° C.

Slurry experiments were conducted at 50° C. in 18 solvents/mixtures. About 50 mg of starting material (Compound I choline salt Form A) was suspended in 0.35-0.5 mL of solvents/mixtures in HPLC vials. After the suspensions were stirred for about 1-6 days at 50° C., the remaining solids were isolated for XRPD analysis. Results are shown in Table 5-9.

TABLE 5-9
Summary of slurry experiments at 50° C.

| Solvent (v:v) | Result |
|---|---|
| IPA | Compound I choline salt Form A |
| 2-Butanol | Compound I choline salt Form A |
| MEK | Compound I choline salt Form A |
| MIBK | Compound I choline salt Form A |
| EtOAc | Compound I choline salt Form A |
| Anisole | Compound I choline salt Form A |
| 2-MeTHF | Compound I choline salt Form A |
| Toluene | Compound I choline salt Form A |
| CHCl₃ | Compound I choline salt Form A |
| ACN/H₂O (19:1) | Compound I choline salt Form A |
| MEK/H₂O (19:1) | Compound I choline salt Form A |
| THF/IPAc (1:1) | Compound I choline salt Form A |
| 1,4-Dioxane/H₂O (19:1) | Compound I choline salt Form A |
| 1,4-Dioxane/n-Heptane (3:1) | Compound I choline salt Form A |
| DMSO/Toluene (1:9) | Compound I choline salt Form A |
| DMAc/CHCl₃ (1:9) | Compound I choline salt Form A |
| NMP/IPAc (1:9) | Compound I choline salt Form A |
| 1,4-Dioxane | Compound I choline salt Form A |

### Slow Cooling

Slow cooling experiments were conducted in eight solvent systems. About 30-40 mg of starting material (Compound I choline salt Form A) was equilibrated in 1.5-3.5 mL of solvents/mixtures at 50° C. for ˜2 hrs in 4-mL glass vials. The solutions/suspensions were filtered using a Nylon membrane (pore size of 0.22 μm) and the filtrates were slowly cooled down to 5° C. at a rate of 0.1° C./min. Results are summarized in Table 5-10.

TABLE 5-10
Summary of slow cooling experiments

| Solvent (v:v) | Result |
|---|---|
| EtOH | Compound I choline salt Form A^{#} |
| ACN | Compound I choline salt Form A* |
| IPA/ACN (1:1) | Gel, orange |
| 2-Butanol/H₂O (19:1) | Compound I choline salt Form A* |
| Acetone/H₂O (19:1) | Compound I choline salt Form A* |
| ACN/Toluene (1:1) | Compound I choline salt Form A* |
| DMSO/IPAc (1:1) | Gel |
| DMSO/H₂O (19:1) | Gel |
| DMAc/1,4-Dioxane (1:2) | Gel |
| DMF/Toluene (2:1) | Compound I choline salt Form A^{#} |
| NMP/Anisole (1:2) | Gel |
| NMP/EtOAc (1:3) | Gel |

Gel, orange: Orange solution was observed after slow cooling (50° C.~5° C.) at 0.1° C./min. Gel was obtained via evaporation after slow cooling and keeping at 5° C./−5° C. for 16 hrs/47 hrs.
Gel: Gel was obtained via vacuum drying at 60° C. after slow cooling followed by keeping at 5° C./−5° C. for 16 hrs/47 hrs and evaporation at RT for 5~11 days.
*Solids were obtained via slow cooling and keeping at 5° C. for 16 hrs.
^{#}Solids were obtained via slow cooling, keeping at 5° C. and −5° C. for 16 hrs, respectively.

### Polymer-Induced Crystallization

About 40˜50 mg of starting material (Compound I choline salt Form A) was dissolved in 1.0˜3.0 mL of solvents in 4-mL glass vials. The solutions/suspensions were filtered using a Nylon membrane (pore size of 0.22 in) and the filtrates were collected. 1˜2 mg polymer mixtures of PVC and PVP were added into the filtrates and the samples were covered by lids with 1 pinhole followed by evaporation at RT. Results are summarized in Table 5-11.

TABLE 5-11
Summary of polymer-induced crystallization experiments

| Solvent (v:v) | Result |
|---|---|
| EtOH | Gel |
| ACN | Gel* |
| MeOH/1,4-Dioxane (1:2) | Compound I choline salt Form A |
| Solvent (v:v) | Result |
| MeOH/CH₂Br₂ (1:2) | Gel* |
| MeOH/EtOAc (1:2) | Compound I choline salt Form A |
| MeOH/2-Butanol (1:2) | Gel |

Polymer: PVP/PVC (1:1, w.w).
*Orange gels were obtained while others presented yellow in color.

About 20 mg of starting material (Compound I choline salt Form A) was grinded with 20˜40 μL corresponding solvents for 3˜5 mins in mortars. Then the solids were characterized via XRPD. Results are summarized in Table 5-12.

TABLE 5-12
Summary of grinding experiments

| Solvent | Result |
|---|---|
| — | Compound I choline salt Form A |
| H₂O | Compound I choline salt Form A |
| MeOH | Compound I choline salt Form A |

### Single Crystal Structure Determination

A suitable single crystal of Compound I choline salt Form A with good diffraction quality was cut out from the block-like crystals and selected for single-crystal X-ray diffraction. The crystal system of the single crystal was monoclinic and the space group was P2₁/c. The unit cell parameters were determined as {a=8.4179(3) Å, b=22.3688(7) Å, c=14.7788(6) Å, α=90°, β=92.803(3)°, γ=90°, V=2779.49(17) ∈³}. Further crystallographic data and the refinement parameters were listed in Table 5-13.

The asymmetric unit of the crystal structure of Compound I choline salt Form A consisted of one free acid anion, one choline cation, and 0.21 water molecule, indicating it was a mono-choline salt. A static disorder of the choline counterpart was observed in the asymmetric unit giving two Parts (Part I and Part II, two conformations of the choline cation). The occupancies, calculated by Olex2, of Part I and Part II were 0.79 and 0.21, respectively. It was noted that a water molecule was co-existed when the choline cation presented as Part II, while no water molecules could be observed in Part I. The occupancy of the water molecule was calculated as 0.21, same as the occupancy of Part II choline cation. It was found that, in Part II, when the choline cation was oriented as conformation II, voids were generated at the positions of water molecules (Calculated by Mercury, probe radius: 1.0 Å, approximate grid spacing: 0.5 Å. Results: void volume of 29.31 Å3. The volume of a water molecule in bulk water was 29.7 Å3. Reference: Gerstein M, Chothia C., Proc Natl Acad Sci., 1996 (19), 10167-10172). In contrast, no void could be found for Part I where the choline cation was oriented as conformation I. Since the percentage of conformation II affected the percentage of water molecules in the crystal lattice, the nature of Compound I choline salt Form A was a non-stoichiometric hydrate. When choline cation was oriented as Part I, the free acid anion and the choline cation was connected together by hydrogen bonding O—H···N. In the case of Part II, a hydrogen bonding interaction of O—H···O was observed instead.

To check whether the water content of Compound I choline salt Form A can be at a lower level, an in-situ TGA heating experiment was conducted. Compound I choline salt Form A showed gradual water loss starting almost from room temperature, characteristics of a non-stoichiometric hydrate. After heating to 180° C. under N₂, in order to remove volatiles, the sample was cooled down for the second round of heating, and a low weight loss of 0.03 wt %, i.e. a molar ratio of water/Compound I of 0.01, was observed. XRPD data of the starting material was collected after heating to 180° C., showing no form change, which was also in good agreement with a non-stoichiometric hydrate.

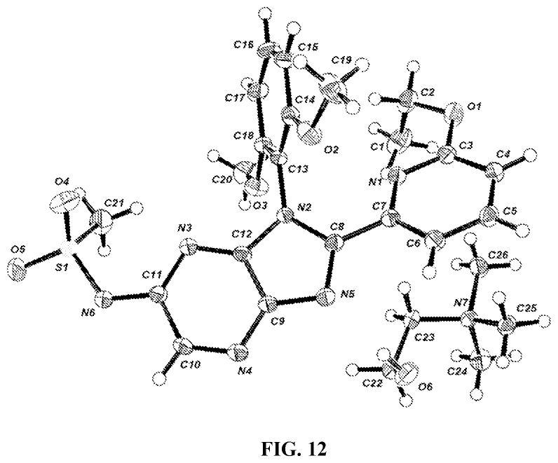

The atomic thermal ellipsoids plot (ORTEP drawing) of the crystal structure of Compound I choline salt Form A is shown in FIG. 12 (displayed as Part1), confirming the proposed chemical structure (atomic thermal ellipsoids were drawn at 50% probability level). In addition, no significant electron density peak could be found around N6 atom, which suggested the acidic proton was transferred, indicating Compound I was a choline salt. This was also supported by the bond lengths of N—S and N—Ar as the deprotonation of R—SO₂—NH—Ar moiety will increase the degree of resonance in both of N—Ar and N—SO₂ functional groups, the bond lengths of N—C and N—S bond will be shortened. The bond lengths of d(N—S) and d(N—C) were 1.5811(12) and 1.3634(19) Å, respectively, which also suggested the deprotonation of N6.

TABLE 5-13
Crystallographic data and refinement parameters
of Compound I choline salt Form A single crystal

| Empirical formula | C₂₆H₃₅N₇O₆S•0.21 H₂O |
|---|---|
| Formula weight | 577.45 |
| Temperature | 113.15 K |
| Wavelength | Mo/Kα (λ = 0.71073 Å) |
| Crystal system, space group | Monoclinic, P2₁/c |
| Unit cell dimensions | a = 8.4179(3) Å |
| | b = 22.3688(7) Å |
| | c = 14.7788(6) Å |
| | α = 90° |
| | β = 92.803(3)° |
| | γ = 90° |
| Volume | 2779.49(17) Å³ |
| Z, Calculated density | 4, 1.380 g/cm³ |
| Absorption coefficient | 0.172 |
| F(000) | 1224.0 |
| Crystal size | 0.22 × 0.18 × 0.14 mm³ |
| 2 Theta range for data collection | 3.642 to 57.4 |
| Limiting indices | −11 ≤ h ≤ 11 |
| | −30 ≤ k ≤ 30 |
| | −19 ≤ 1 ≤ 19 |
| Reflections collected/Independent reflections | 31916/7184 |
| Refinement method | Full-matrix least-squares on F² |
| Completeness | 100% |
| Data/restrains/parameters | 7184/14/444 |
| Goodness-of-fit on F² | 1.032 |
| Final R indices [I ≥ 2 σ (I)] | R₁ = 0.0441, wR₂ = 0.1037 |
| Final R indices [all data] | R₁ = 0.0598, wR₂ = 0.1139 |
| Largest diff. peak and hole | 0.32/−0.49 |

TABLE 5-14
H-bond list in Compound I choline salt Form A single crystal structure

| Form | D-H . . . A | d(D-H)/Å | d(H-A)/Å | d(D . . . A)/Å | (D-H . . . A)/° |
|---|---|---|---|---|---|
| Intermolecular¹ | O6-H6a . . . N5 | 0.87(3) | 2.02(4) | 2.8672(19) | 163(3) |
| (Part I) | | | | | |
| Intermolecular² | O6a-H6aa . . . O4 | 0.839 | 1.674 | 2.922 | 138.58 |
| (Part II) | | | | | |

¹Calculated by PLATON program (version: 260918), (Analysis of Potential Hydrogen Bonds with d(D . . . A) < R(D) + R(A) + 0.50 Ang., d(H . . . A) < R(H) + R(A) − 0.12 Ang., D-H . . . A) > 100.0 Deg)
²Measured by Mercury

Based on the characterization results, Compound I choline salt Form A was obtained with its potential single crystals observed. The single crystal structure determination confirmed the chemical structure of Compound I choline salt, showing Form A was a non-stoichiometric hydrate (channel hydrate) of mono-choline salt.

### Example 6: Dog Pharmacokinetic Studies for Compound I Choline Salt Form A and Free Acid Form A

The objective of this study was to characterize the pharmacokinetics (PK) of Compound I in male Beagle dogs after oral (P0) administration. The P0 formulation was prepared in capsule or prepared in 0.50% or 100 MC (methyl cellulose) in water at a concentration of 6 mg/mL for oral administration at 30 mg/kg.

### Instruments and Methods

LC-MS/MS for the PK studies was carried out using the following instruments/conditions.

TABLE 6-1
LC-MS/MS conditions used

| LC-MS/MS Instrument | | |
|---|---|---|
| Autosampler | SIL-30AC (Shimadzu, Japan) | |
| HPLC equipment | LC-30AD (Shimadzu, Japan) | |
| MS/MS instrument | Triple Quad 6500+ (AB Sciex, USA) | |
| LC-MS/MS Conditions | | |
| Column | Waters X-Bridge BEH C18 | |
| | (2.1 × 50 mm, 2.5 μm) | |
| Mobile Phase | A: H₂O-0.025% FA-1 mM NH₄OAc | |
| | B: ACN-0.025% FA-1 mM NH₄OAc | |
| | Time (min) | % B |
| | 0.30 | 2 |
| | 1.00 | 90 |
| | 1.50 | 90 |
| | 1.51 | 2 |
| | 2.00 | Stop |

| | |
|---|---|
| Flow rate | 0.80 mL/min |
| Column Temperature | 50° C. |
| Injection Volume | 1 μL |
| Ion Source | Turbo Spray |
| Ionization Model | ESI |
| Scan Type | MRM |
| Ionization Mode | Positive |

### Dose Formulation

Compound I Free acid Form A and Compound I choline salt Form A were formulated as an oral suspension at a target dose concentration of 6 mg/mL in 0.500 or 1% MC in water.

The dose was administered orally by gavage at a target dose volume of 5 mL/kg. Compound I choline salt Form A was also formulated as an oral capsule at a target dose concentration of 30 mg/kg. The formulation was prepared on the day of dosing and stored at room temperature prior to administration. The dose was administered orally by gavage to dogs. Following oral administration, blood samples were collected from the oral gavage at pre-dose, 0.083, 0.25, 0.5, 1, 2, 4, 8 and 24 h post dose.

The plasma concentration of Compound I was determined by liquid chromatography with mass spectrometric detection (LC-MS/MS).

### Results

The pharmacokinetic data are summarized in Table 6-3 below.

TABLE 6-3
Summary of Mean PK parameters of Compound I

| | | Cₘₐₓ | Tₘₐₓ | AUCₗₐₛₜ |
|---|---|---|---|---|
| Polymorph | Dose | (ng/mL) | (h) | (h*ng/mL) |
| Free acid | Suspension, | 983 | 4.0 | 13819 |
| Form A | 30 mg/kg | | | |
| Choline salt | Suspension, | 73850 | 2.0-4.0 | 1115424 |
| Form A | 30 mg/kg | | | |
| Choline salt | Capsule, | 11915 | 4.0 | 145309 |
| Form A | 30 mg/kg | | | |

AUCₗₐₛₜ t = 24 h for PO administration.
Tₘₐₓ was presented as median value or range if mean value is not applicable.

### Conclusion

Compound I choline salt Form A in both suspension and capsule showed excellent pharmacokinetic profiles. The mean AUCₗₐₛₜ values and Cₘₐₓ values of Compound I choline salt Form A, in either suspension or capsule formulation, were greater than that of Compound I free acid Form A administered as a suspension. Thus Compound I choline salt Form A demonstrated better oral bioavailability over Compound I free acid Form A.

One skilled in the art would readily appreciate that the present disclosure is well adapted to obtain the ends and advantages mentioned, as well as those inherent therein. The methods, variances, and compositions described herein as presently representative of embodiments are exemplary and are not intended as limitations on the scope of the disclosure. Changes therein and other uses will occur to those skilled in the art, which are encompassed within the spirit of the disclosure, are defined by the scope of the claims.

1. (canceled)

2. A crystalline form of N-(1-(2,6-dimethoxyphenyl)-2-(6-ethoxypyridin-2-yl)-1H-imidazo[4,5-b]pyrazin-6-yl)methanesulfonamide choline salt (Compound I choline salt), or solvate thereof, having the formula:

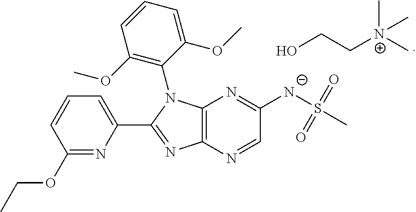

3. Crystalline N-(1-(2,6-dimethoxyphenyl)-2-(6-ethoxypyridin-2-yl)-1H-imidazo[4,5-b]pyrazin-6-yl)methanesulfonamide choline salt Form A (Compound I choline salt Form A), characterized by an X-ray powder diffractogram comprising the following peaks expressed in ±0.2 degrees 2-theta selected from: 12.9, 14.6, and 18.1, as determined on a diffractometer using Cu-Kα radiation.

4. The crystalline Compound I choline salt Form A of claim 3, further characterized by an X-ray powder diffractogram comprising one or more additional peaks expressed in ±0.2 degrees 2-theta selected from: 9.9, 16.7, 17.5, 19.8, 22.7, and 23.1, as determined on a diffractometer using Cu-Kα radiation.

5. The crystalline Compound I choline salt Form A of claim 3, further characterized by an X-ray powder diffractogram as substantially shown in FIG. 1A.

6. The crystalline Compound I choline salt Form A of claim 3, further characterized by a DSC comprising a endotherm at about 194-200 (peak).

7. The crystalline Compound I choline salt Form A of claim 3, further characterized by a DSC as substantially shown in FIG. 1B.

8. Crystalline N-(1-(2,6-dimethoxyphenyl)-2-(6-ethoxypyridin-2-yl)-1H-imidazo[4,5-b]pyrazin-6-yl)methanesulfonamide choline salt Form A (Compound I choline salt Form A), having unit cell parameters: α=8.4179(3) Å, b=22.3688(7) Å, c=14.7788(6) Å, α=90°, β=92.803(3)°, γ=90°, V=2779.49(17) ∈³.

9. Crystalline N-(1-(2,6-dimethoxyphenyl)-2-(6-ethoxypyridin-2-yl)-1H-imidazo[4,5-b]pyrazin-6-yl)methanesulfonamide choline salt Form B (Compound I choline salt Form B), characterized by an X-ray powder diffractogram comprising the following peaks expressed in ±0.2 degrees 2-theta selected from: 11.0, 14.5, and 19.2, as determined on a diffractometer using Cu-Kα radiation.

10. The crystalline Compound I choline salt Form B of claim 9, further characterized by an X-ray powder diffractogram comprising one or more additional peaks expressed in ±0.2 degrees 2-theta selected from: 18.4, 18.7, 19.8, 21.6, 22.1, and 24.4, as determined on a diffractometer using Cu-Kα radiation; an X-ray powder diffractogram as substantially shown in FIG. 2A; a DSC comprising a peak at 74.4° C. (peak) and 172.1° C. (peak); or a DSC as substantially shown in FIG. 2B.

11. Crystalline N-(1-(2,6-dimethoxyphenyl)-2-(6-ethoxypyridin-2-yl)-1H-imidazo[4,5-b]pyrazin-6-yl)methanesulfonamide Form B (Compound I free acid Form B), characterized by an X-ray powder diffractogram comprising the following peaks expressed in ±0.2 degrees 2-theta selected from: 9.5, 13.8, and 15.2, as determined on a diffractometer using Cu-Kα radiation.

12. The crystalline Compound I free acid Form B of claim 11, further characterized by an X-ray powder diffractogram comprising one or more additional peaks expressed in ±0.2 degrees 2-theta selected from: 14.2, 17.1, 19.6, 26.0, 26.9, and 27.9°2θ±0.2°2θ, as determined on a diffractometer using Cu-Kα radiation; an X-ray powder diffractogram as substantially shown in FIG. 4A; a DSC comprising a peak at 108.8° C. (peak) and 230.4° C. (peak); or a DSC as substantially shown in FIG. 4B.

13. A crystalline salt form of N-(1-(2,6-dimethoxyphenyl)-2-(6-ethoxypyridin-2-yl)-1H-imidazo[4,5-b]pyrazin-6-yl)methanesulfonamide (Compound I), or solvate thereof, having the Formula: 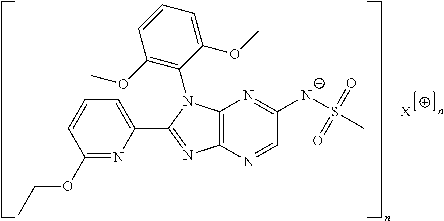
wherein: X is sodium and n is 1; X is potassium and n is 1, X is calcium and n is 2, or X is magnesium and n is 2.

14. The crystalline salt form of claim 13, wherein the crystalline salt form is selected from the group consisting of: Compound I sodium salt Form A, Compound I potassium salt Form A, Compound I potassium salt Form B, Compound I calcium salt Form A, and Compound I magnesium salt Form A.

15. A pharmaceutical composition comprising the crystalline form of Compound I, choline salt of claim 2, and a pharmaceutically acceptable excipient.

16. (canceled)

17. The pharmaceutical composition of claim 15, wherein at least 99% of Compound I in the pharmaceutical composition is the crystalline form of the Compound I choline salt, or solvate thereof, of claim 2.

18.-20. (canceled)

21. A method for treating a disease, disorder, or condition, in which repressed or impaired APJ receptor signaling, or downregulation of endogenous apelin contributes to the pathology and/or symptoms and/or progression of the disease, disorder, or condition, comprising administering to a subject in need thereof an effective amount of the pharmaceutical composition of claim 15.

22. The method of claim 21, wherein the disease, disorder, or condition, is pulmonary arterial hypertension (PAH).

23. The method of claim 22, wherein the PAH is idiopathic.

24. The method of claim 22, wherein the PAH is heritable PAH, toxin or drug-induced PAH; or a PAH associated with one or more of congenital heart disease, connective tissue disorders (e.g., scleroderma, systemic lupus erythematosus, systemic sclerosis, Hashimoto's thyroiditis, Sjogren's Syndrome, and the antiphospholipid antibody syndrome), portal hypertension, BMPR2 mutations, Schistosomiasis, or HIV infection.

25. The method of claim 21, wherein the disease, disorder, or condition, is fibrosis.

26. The method of claim 25, wherein the fibrosis is associated with an organ or tissue selected from the group consisting of lung, liver, heart, mediastinum, bone marrow, retroperitoneum, skin, intestine, joint, and a reproductive organ, or a combination thereof.

27. The method of claim 21, wherein the disease, disorder, or condition is idiopathic pulmonary fibrosis (IPF).

28. The method of claim 21, wherein the disease, disorder, or condition, is a connective tissue disorder.

29. The method of claim 28, wherein the connective tissue disorder is selected from the group consisting of scleroderma, systemic lupus erythematosus, systemic sclerosis, Hashimoto's thyroiditis, Sjogren's Syndrome, and antiphospholipid antibody syndrome.

30. The method of claim 21, wherein the disease, disorder, or condition is systemic sclerosis.

31. A process for preparing crystalline N-(1-(2,6-dimethoxyphenyl)-2-(6-ethoxypyridin-2-yl)-1H-imidazo[4,5-b]pyrazin-6-yl)methanesulfonamide choline salt (Compound I choline salt) comprising contacting Compound I, or a salt thereof, with choline in a solvent for a time sufficient to provide a crystalline Compound I choline salt.

32.-36. (canceled)
